(19) United States
(12) Patent Application Publication
(10) Pub. No.: US 2025/0090595 A1
(43) Pub. Date: Mar. 20, 2025

(54) THERAPEUTIC USE OF CANCER-ASSOCIATED FIBROBLAST-ENCAPSULATED PANCREATIC BETA CELLS FOR TREATING DIABETES

(71) Applicants: Hamid KHOJA, Houston, TX (US); Pete O'HEERON, Houston, TX (US); Nikolay BAZHANOV, Houston, TX (US)

(72) Inventors: Hamid KHOJA, Houston, TX (US); Pete O'HEERON, Houston, TX (US); Nikolay BAZHANOV, Houston, TX (US)

(21) Appl. No.: 18/884,869

(22) Filed: Sep. 13, 2024

Related U.S. Application Data
(60) Provisional application No. 63/583,158, filed on Sep. 15, 2023.

(51) Int. Cl. A61K 35/39; A61K 35/33; A61K 47/69; A61P 3/10

(52) U.S. Cl. CPC A61K 35/39; A61K 35/33; A61K 47/6911; A61P 3/10

(57) ABSTRACT

Disclosed herein are compositions comprising one or more cell types encapsulated by a plurality of cancer-associated fibroblasts (CAFs) and/or fibroblasts; and/or one or more cell types encapsulated by extracellular matrix (ECM) secreted by CAFs and/or fibroblasts. Also disclosed are methods for producing compositions disclosed herein, methods of using compositions disclosed herein for treatment of diseases and/or disorders, and kits for practicing the methods disclosed herein.

### CROSS-REFERENCE TO RELATED APPLICATION

This application claims priority to U.S. Provisional Patent Application Ser. No. 63/583,158, filed Sep. 15, 2023, which is incorporated by reference herein in its entirety.

### I. TECHNICAL FIELD

Aspects of the disclosure concern at least the fields of cell biology, molecular biology, immunology, and medicine.

### II. BACKGROUND

Diabetes mellitus is a chronic heterogeneous metabolic disease characterized by elevated blood glucose levels due to abnormalities in either insulin production or insulin action that impacts more than 37 million people in the United States, and over 500 million people worldwide [1, 2]. Type 1 diabetes, an autoimmune disorder in which the immune system destroys the pancreatic beta cells which produce insulin, accounts for 10% of diabetes cases worldwide, and type 2 diabetes in which the body does not respond to insulin, accounts for the remaining 90% of diabetes cases [3, 4]. With such a high and growing prevalence of diabetes, over 200 million individuals are dependent on consistent, and often daily insulin therapy [5]. Although transplantation of pancreatic islets has been proposed as a therapy against diabetes, this approach remains unsuitable due to the effects of the immune system. Immediately after transplantation, about 25% of transplanted islets are destroyed by the immune system (Welsch C A, Rust W L, Csete M. Concise Review: Lessons Learned from Islet Transplant Clinical Trials in Developing Stem Cell Therapies for Type 1 Diabetes. Stem Cells Transl Med. 2019 March; 8 (3): 209-214). At later stages, immune rejection mechanisms continue to reject the transplanted islets (Kale A, Rogers N M. No Time to Die-How Islets Meet Their Demise in Transplantation. Cells. 2023 Mar. 3; 12 (5): 796).

There is an unfulfilled need for long-term, economical, and effective treatment options for diabetes.

### SUMMARY

The present disclosure provides compositions and methods for encapsulating cells (e.g., insulin-producing cells) using cancer-associated fibroblasts (CAFs), fibroblasts, fibroblast-secreted ECM (FECM), and/or CAF-secreted extracellular matrix (CECM). Encapsulation of cells by CAFs, fibroblasts, FECM, and/or CECM, can protect the encapsulated cells from targeting, attack, and/or killing by the immune system. In addition, the encapsulation by CAFs, CAFs with CEMC, fibroblasts, FECM, and/or CECM can provide a source of anchoring to the transplanted tissue (e.g., beta cells), and may promote blood vessel formation to improve nutrition supply to the transplanted tissue. The cells may be encapsulated in spheroids and/or organoids comprising CAFs, CEMC, fibroblasts, and/or FECM.

In some aspects, the encapsulated cells comprise, consist of, or consist essentially of insulin-producing cells, e.g., pancreatic beta cells. In some aspects, the encapsulated cells secrete a desired protein, enzyme, or metabolite, e.g., a desired extracellular matrix protein secreted by a fibroblast or CAF. In some aspects, encapsulated beta cells are protected from the immune system to enable long-term and stable in vivo persistence and production of insulin in a diabetic patient. In some aspects, a number of encapsulated cells may be about 1 to 10,000 cells. In some aspects, a number of encapsulated cells may be about 1 to 100,000 cells.

In some aspects, CAFs and/or fibroblasts are engineered using gene editing to secrete ECM. In some aspects, CAFs and/or fibroblasts are engineered with CRISPR to secrete ECM. In some aspects, CAFs and/or fibroblasts are engineered using gene editing to secrete ECM when activated in vivo. In some aspects, CAFs and/or fibroblasts are engineered using CRISPR to secrete ECM when activated in vivo.

In some aspects, a composition of the disclosure may further comprise further comprising one or more fibroblast-derived and/or CAF-derived materials. In some aspects, fibroblast-derived and/or CAF-derived materials comprise exosomes, lysates, membranes, apoptotic bodies, or a mixture thereof.

Some aspects relate to methods of generating one or more compositions of the disclosure. In some aspects, a method for encapsulating cells comprises generating one or more spheroids from the cells to be encapsulated in a pipette tip, microwell, or microcavity, for example. In some aspects, a method may comprise withdrawing CAFs, fibroblasts, FECM, and/or ECM into a pipette tip, microwell, or microcavity comprising spheroids of cells to be encapsulated. In some aspects, the CAFs and/or fibroblasts used in methods or compositions of the disclosure comprise, consist of, or consist essentially of allogeneic cells, autologous cells, syngeneic cells, xenogeneic cells, or a mixture thereof with respect to an individual. In some aspects, the cells encapsulated by the CAFs and/or fibroblasts used in methods or compositions of the disclosure comprise, consist of, or consist essentially of allogeneic cells, autologous cells, syngeneic cells, xenogeneic cells, or a mixture thereof with respect to an individual.

In some aspects, CAFs and/or fibroblasts used in in methods or compositions of the disclosure may be treated ex vivo to secrete ECM prior to, during, and/or following generation of a composition of the disclosure. In some aspects, CAFs and/or fibroblasts used in methods or compositions of the disclosure may be treated with one or more of hypoxic conditions, oxidative stress, and/or one or more growth factors produced by tumor cells. In some aspects, a growth factor comprises, consists of, or consists essentially of one or more of TGF-β, epidermal growth factor (EGF), fibroblast growth factor type 2 (FGF2), PDGF, Activin A, Nodal, or one or more BRAF inhibitors. In some aspects, a BRAF inhibitor comprises, consists of, or consists essentially of Vemurafenib, dabrafenib, encorafenib, or a combination thereof. In some aspects, CAFs and/or fibroblasts used in methods or compositions of the disclosure may be activated prior to, during, and/or following generation of a composition of the disclosure with cytokines, chemokines, growth factors, transcription factors, and/or nucleic acids to secrete ECM. In some aspects, CAFs and/or fibroblasts used in methods or compositions of the disclosure may be activated prior to, during, and/or following generation of a composition of the disclosure with CAF-derived and/or fibroblast-derived materials. In some aspects, CAF-derived and/or fibroblast-derived materials comprise, consist of, or consist essentially of exosomes, lysates, extracellular vesicles, membranes or a combination thereof. In some aspects, CAFs and/or fibroblasts are activated to secrete ECM.

In some aspects, CAFs and/or fibroblasts used in methods or compositions of the disclosure may be engineered prior to, during, and/or following generation of a composition of the disclosure. In some aspects, CAFs and/or fibroblasts are engineered to secrete ECM.

In some aspects, CAFs and/or fibroblasts are derived from skin, or pancreas of an individual. In some aspects, fibroblasts of the disclosure comprise human dermal fibroblasts (HDF), and/or human neonatal fibroblasts.

Some aspects of the disclosure concern methods of administering compositions of the disclosure to an individual in need thereof. In some aspects, compositions of the disclosure may be administered to an individual in need thereof by injectable insertion. In some aspects, encapsulated cells administered to an individual in need thereof comprise pancreatic beta cells. In some aspects, an individual in need thereof has been diagnosed, is suspected of having, or shows symptoms of having diabetes. In some aspects, an individual in need thereof has been diagnosed, is suspected of having, or shows symptoms of having a metabolic disease, a genetic disorders, or a combination thereof that results in a lack of production of sufficient amounts of a functional protein and/or enzyme. In some aspects, an individual in need thereof has been diagnosed, is suspected of having, or shows symptoms of having one or more metabolic disorders, such as phenylketonuria, tyrosinemia, homocystinuria, non-ketotic hyperglycinemia and maple syrup urine disease. other examples are amyloidogenic disorders such as inherited cataracts, some forms of atherosclerosis, hemodialysis-related disorders, and short-chain amyloidosis syndrome. Other examples are achondroplasia, Morquio A syndrome, mucopolysaccharidosis I, CLN2 disease, maroteaux-Lamy syndrome, Alternating hemiplegia of childhood (AHC), Hydrops ectopic calcification-moth-caten (HEM), Alzheimer's disease, Parkinson's disease, Huntington's disease, Creutzfeldt-Jakob disease, cystic fibrosis, Gaucher's disease, Tay-sachs, or Fanconi anemia.

In some aspects, encapsulated cells can be injected or transplanted into one or more tissues, such as the liver, portal vein, pancreas, sub-dermal layer of skin, intraperitoneal, and intramuscular regions. In some aspects, encapsulated cells engraft into the injected or transplanted tissue and provide a long-term supply of a desired therapeutic product, such as insulin, to the individual.

Some aspects of the disclosure relate to a kit, housed in a suitable contained, comprising, consisting of, or consisting essentially of one or more of the compositions disclosed herein. In some aspects, a kit further comprises one or more apparatuses to generate and/or administer one or more of the compositions disclosed herein. In some aspects, a kit comprises a pipettor, pipette tips, and/or a syringe.

It is specifically contemplated that any limitation discussed with respect to one aspect of the invention may apply to any other aspect of the invention. Furthermore, any composition of the invention may be used in any method of the invention, and any method of the invention may be used to produce or to utilize any composition of the invention. Any aspect discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an aspect set forth in the Examples are also aspects that may be implemented in the context of aspects discussed elsewhere in a different Example or elsewhere in the application, such as in the Summary, Detailed Description, Claims, and Brief Description of the Drawings.

It is to be understood that the present disclosure is not limited to particular aspects described, as such may, of course, vary. It is also to be understood that the terminology used herein is for the purpose of describing particular aspects only, and is not intended to be limiting, since the scope of the present disclosure will be limited only by the appended claims.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The terms “comprise” (and any form of comprise, such as “comprises” and “comprising”), “have” (and any form of have, such as “has” and “having”), and “include” (and any form of include, such as “includes” and “including”) are open-ended linking verbs. As a result, a composition that “comprises,” “has,” or “includes” one or more elements possesses those one or more elements, but is not limited to possessing only those elements. Likewise, a method that “comprises,” “has,” or “includes” one or more steps possesses those one or more steps, but is not limited to possessing only those one or more steps.

Any configuration of any compositions or methods can consist of or consist essentially of—rather than comprise/include/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb. The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

The term “engineered” as used herein refers to an entity that is generated by the hand of man, including a cell, nucleic acid, polypeptide, vector, and so forth. In at least some cases, an engineered entity is synthetic and comprises elements that are not naturally present or naturally configured in comparison to the manner in which it is utilized in the disclosure. In specific aspects, a vector is engineered through recombinant nucleic acid technologies, and a cell is engineered, e.g., through transfection or transduction of an engineered vector. Cells may be engineered to express heterologous proteins that are not naturally expressed by the cells, either because the heterologous proteins are recombinant or synthetic or because the cells do not naturally express the proteins.

“Individual,” “subject,” and “patient” are used interchangeably and can refer to a human or non-human, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. An individual may or may not be housed in a medical facility and may be treated as an outpatient of a medical facility. The individual may be receiving one or more medical compositions. An individual may comprise any age of a human or non-human animal and therefore includes both adults and juveniles (i.e., children) and infants. It is not intended that the term “individual” connotes a need for medical treatment, therefore, an individual may voluntarily or involuntarily be part of experimentation whether clinical or in support of basic science studies.

As used herein, the term “cancer associated fibroblast” refers to a fibroblasts found within the tumor or tumor microenvironment.

As used herein, the term “organoid” is used interchangeably with “spheroids” and refers to three-dimensional organization of cells. An organoid or spheroid may comprise more than one cell type and each cell type may be arranged in a specific manner. For example, an organoid or spheroid may comprise two or more layers of different cell types. An inner layer of cells may be referred to as the “core” of an organoid or spheroid. One or more layers of cells surrounding the core of an organoid or spheroid may be referred to as the “shell.” An organoid may have multiple shell layers, each of which may be composed of different cell types or the same cell types.

As used herein, the term “encapsulated” refers to enveloping or covering a plurality of cells with a plurality of other cells. The encapsulated cells may be a different cell type than the cells by which they are encapsulated.

As used herein, the term “activated cells” refers to cells treated with one or more stimuli capable of inducing one or more alterations in the cell: metabolic, immunological, epigenetic, growth factor secreting, surface marker expression, and production and excretion of proteins or microvesicles (e.g., exosomes).

The term “administered” or “administering”, as used herein, refers to any method of providing a composition to an individual such that the composition has its intended effect on the individual. For example, one method of administering is by an indirect mechanism using a medical device such as, but not limited to a catheter, applicator gun, syringe, etc. Another method of administering may be by a direct mechanism such as, local tissue administration, implantation, ingestion, transdermal patch, topical, inhalation, suppository, etc.

As used herein, “allogeneic” refers to tissues or cells from another body that in a natural setting are immunologically incompatible or capable of being immunologically incompatible, although from one or more individuals of the same species (e.g., allogeneic pancreatic beta cells).

As used herein, “autologous” refers to tissues or cells that are derived or transferred from the same individual's body (e.g., cells derived from an autologous biopsy).

As used herein, “agent” refers to nucleic acids, cytokines, chemokines, transcription factors, epigenetics factors, growth factors, or hormones.

As used herein, “xenogeneic” refers to tissues or cells from a species different from the patient.

As used herein, “cell culture” refers to an artificial in vitro system containing viable cells, whether quiescent, senescent, or actively dividing. In a cell culture, cells are grown and maintained at an appropriate temperature, typically a temperature of about 37° C. and under an atmosphere typically containing oxygen and CO₂. Culture conditions may vary widely for each cell type, and variation of conditions for a particular cell type can result in different phenotypes. The most commonly varied factor in culture systems is the growth medium. Growth media can vary in concentration of nutrients, growth factors, and the presence of other components. The growth factors used to supplement media are often derived from animal blood, such as calf serum.

Reference throughout this specification to “one aspect,” “an aspect,” “a particular aspect,” “a related aspect,” “a certain aspect,” “an additional aspect,” or “a further aspect” or combinations thereof means that a particular feature, structure or characteristic described in connection with the aspect is included in at least one aspect of the present disclosure. Thus, the appearances of the foregoing phrases in various places throughout this specification are not necessarily all referring to the same aspect. Furthermore, the particular features, structures, or characteristics may be combined in any suitable manner in one or more aspects.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one aspect, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

As used herein, the term “transplantation” refers to the process of taking living tissue or cells and implanting it in another part of the body or into another body.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the concentration of sugar in the blood of an individual is considered to be a treatment if there is a detectable reduction in the concentration of sugar in the blood of the individual when compared to pre-treatment levels in the same subject or control subjects. Thus, the reduction can be a 10, 20, 30, 40, 50, 60, 70, 80, 90, 100%, or any amount of reduction in between as compared to control levels. It is understood and herein contemplated that “treatment” does not necessarily refer to a cure of the disease or condition, but an improvement in the outlook of a disease or condition. In specific aspects, treatment refers to the lessening in severity or extent of at least one symptom and may alternatively or in addition refer to a delay in the onset of at least one symptom.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific aspects presented herein.

FIG. 1 shows spheroids comprising fibroblast cells alone, beta cells alone, or a mixture of beta cells to fibroblasts at 1:4, 1:1, and 4:1 ratios.

FIG. 2 shows representative immunofluorescence images of nuclei (DAPI), beta cells (insulin), and fibroblasts (vimentin) from spheroids comprising fibroblast cells alone, beta cells alone, or a 1:1 mixture of fibroblasts and beta cells.

FIG. 3 shows insulin production by spheroids comprising fibroblast cells alone, beta cells alone, or a 1:4, 1:1, or 4:1 mixture of beta cells to fibroblasts in control and glucose stimulated conditions.

FIG. 4 shows insulin production by spheroids comprising fibroblast cells alone, beta cells alone, or a 1:4, 1:1, or 4:1 mixture of beta cells to fibroblasts after exposure to innate immune cells.

### DETAILED DESCRIPTION

This application incorporates by reference herein U.S. Provisional Patent Application Ser. No. 63/583,158, filed Sep. 15, 2023.

The practice of the present disclosure may employ, unless otherwise indicated, conventional techniques of tissue culture, immunology, molecular biology, microbiology, cell biology and recombinant DNA, which are within the skill of the art. See, e.g., Green and Sambrook eds. (2012) Molecular Cloning: A Laboratory Manual, 4th edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (IRL Press (1986)); Grandi cd. (2007) In Vitro Transcription and Translation Protocols, 2nd edition; Guisan ed. (2006) Immobilization of Enzymes and Cells; Perbal (1988) A Practical Guide to Molecular Cloning, 2nd edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4th edition; and Herzenberg et al. eds (1996) Weir's Handbook of Experimental Immunology, 5th edition.

It also is to be understood, although not always explicitly stated, that the reagents described herein are merely illustrative and that equivalents of such are known in the art. It is to be inferred without explicit recitation and unless otherwise intended, that when the present technology relates to a nucleic acid, protein, polynucleotide, cell, or antibody, an equivalent or a biologically equivalent of such is intended within the scope of the present technology.

Unless defined otherwise, all technical and scientific terms used herein have the same meanings as commonly understood by one of ordinary skill in the art to which this technology belongs. Although any methods and materials similar or equivalent to those described herein can be used in the practice or testing of the present technology, the preferred methods, devices and materials are now described. All technical and patent publications cited herein are incorporated herein by reference in their entirety.

Aspects of the disclosure encompass improvements to cell engraftment (including allogeneic cell engraftment) in an individual, including human, dog, cat, horse, and so forth. Improvements include at least the ability to have greater immunotolerance in vivo for the grafted cell population administered to the individual. Aspects of the disclosure include a novel method for encapsulating cells using cancer associated fibroblasts (CAFs), CAF-secreted extracellular matrix (CECM), or a combination thereof. Encapsulation of cells by CAFs and/or CECM can protect the encapsulated cells from detection and destruction by the immune system. When the encapsulated cells are secretory cells (e.g., insulin-producing cells, or therapeutic agent-producing cells), the encapsulated cells can persist in the individual longer than non-encapsulated cells to produce sufficient amounts of a therapeutic agent (e.g., insulin).

### A. Fibroblasts and Cancer-Associated Fibroblasts

Cancer-associated fibroblasts (CAFs) have become the target of academic and industry cancer researchers for their role in protecting tumors from the immune system by promoting tumor vascularization, growth, and metastasis, and severely impacting the efficacy of chemotherapeutic and immunotherapeutic treatments [6-11]. CAFs have been identified in many cancer types and their presence and prevalence has been linked to changes in prognosis and responses to therapy [12]. Research has indicated that CAFs achieve this through modulation of the immune response and secretion of an extracellular matrix (ECM) that encapsulates tumors and protect them from immune responses and anti-cancer therapies [13-17]. To date, efforts have focused on developing treatments for preventing the protective effects of CAFs to more effectively treat cancer. However, the present disclosure presents a novel use for CAFs in which a cell of interest, for example an allogeneic insulin-secreting cell, is protected from the immune system of a recipient individual.

### 1. Isolation

The CAFs and/or fibroblasts considered in this disclosure may be generated, in one or more aspects, by outgrowth from a biopsy of an individual (e.g., an individual with cancer). CAFs and/or fibroblasts may be from an allogeneic, syngeneic, xenogeneic, autologous source, or a mixture thereof with respect to an individual. Any individual with cancer may be a source of CAFs, including infants, children, adolescents, and/or adults.

CAFs may be isolated from a cancer that originated from bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus tissue in an individual, or from tissue adjacent and/or surrounding the cancer, such as the tumor microenvironment. Fibroblasts may be isolated from bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus tissue from an individual.

The CAFs and/or fibroblasts may be obtained by any suitable manner known in the art. As an example, about 1-50 mm³, or about 1-500 milligrams, of tissue are acquired by biopsy from an individual (e.g., an individual with cancer). The biopsy may be collected using standard practices, such as core needle biopsy, surgical biopsy, and/or fine needle biopsy. The biopsies may be placed into a vial containing sterile phosphate buffered saline (PBS) and shipped in a 2-8° C. refrigerated shipper back to a manufacturing or processing facility. After arrival at the facility, the biopsy may be inspected and, upon acceptance, transferred directly to a processing area.

CAFs and/or fibroblasts may be isolated from a sample or biopsy of bodily tissue by enzymatic digestion, mechanical separation, filtration, centrifugation, or combinations thereof. The number and quality of the isolated CAFs and/or fibroblast cells can vary depending, e.g., on the quality of the tissue used, the compositions of perfusion buffer solutions, and the type and concentration of enzymes. Frequently used enzymes include, but are not limited to, collagenase, pronase, trypsin, dispase, hyaluronidase, thermolysin and pancreatin, and combinations thereof. Collagenase is most commonly used, often prepared from bacteria (e.g., from Clostridium histolyticum), and may often consist of a poorly purified blend of enzymes, which may have inconsistent enzymatic action. Some of the enzymes exhibit protease activity, which may cause unwanted reactions affecting the quality and quantity of viable/healthy CAF and/or fibroblast cells. It is understood by those of skill in the art to use enzymes of sufficient purity and quality to obtain viable CAF and/or fibroblast cell populations.

In some aspects, a collagenase enzyme solution (e.g., Liberase™) is added to the tissue sample without mincing and incubated at 37.0±2° C. for about 1-4 hours. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.), for example. Alternatively, other commercially available collagenases may be used, such as Serva Collagenase NB6 (Heidelberg, Germany). After digestion, Initiation Growth Media (IMDM, GA, 10% Fetal Bovine Serum (FBS)) is added to neutralize the enzyme, cells are pelleted by centrifugation and re-suspended in 5.0 mL Initiation Growth Media. Alternatively, centrifugation is not performed, with full inactivation of the enzyme occurring by the addition of Initiation Growth Media only. Initiation Growth Media is added prior to seeding of the cell suspension into a T-175 cell culture flask for initiation of cell growth and expansion. A T-75, T-150, T-185 or T-225 flask can be used in place of the T-175 flask. Cells are incubated at 37±2.0° C. with 5.0±1.0% CO₂ and fed with fresh Complete Growth Media every three to five days. All feeds in the process are performed by removing half of the Complete Growth Media and replacing the same volume with fresh media. Alternatively, full feeds can be performed. Cells should not remain in the T-175 flask greater than 30 days prior to passaging. Confluence is monitored throughout the process to ensure adequate seeding densities during culture splitting. When cell confluence is greater than or equal to 40% in the T-175 flask, they are passaged by removing the spent media, washing the cells, and treating with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask. Cells may be incubated at 37±2.0° C. with 5.0±1.0% CO₂ and passaged every three to five days in the T-500 flask and every five to seven days in the ten layer cell stack (10 CS). Cells should not remain in the T-500 flask for more than 10 days prior to passaging. Quality Control (QC) release testing for safety includes sterility and endotoxin testing. When cell confluence in the T-500 flask is about 95%, cells are passaged to a 10 CS culture vessel. Alternately, two Five Layer Cell Stacks (5 CS) or a 10 Layer Cell Factory (10 CF) can be used in place of the 10 CS. Passage to the 10 CS is performed by removing the spent media, washing the cells, and treating with Trypsin-EDTA to release adherent cells in the flask into the solution. Cells are then transferred to new 10 CS. Additional Complete Growth Media is added to neutralize the trypsin and the cells from the T-500 flask are pipetted into a 2 L bottle containing fresh Complete Growth Media. The contents of the 2 L bottle are transferred into the 10 CS and seeded across all layers. Cells are then incubated at 37±2.0° C. with 5.0±1.0% CO₂ and fed with fresh Complete Growth Media every five to seven days. Cells should not remain in the 10 CS for more than 20 days prior to passaging. Prior to use for therapeutic purposes, CAFs are rendered substantially free of immunogenic proteins present in the culture medium by incubating the CAFs for a period of time in protein free medium. When cell confluence in the 10 CS is about 95% or more, cells may be harvested for use in any of the methods described herein. Harvesting is performed by removing the spent media, washing the cells, treating with Trypsin-EDTA to release adherent cells into the solution, and adding additional Complete Growth Media to neutralize the trypsin. Cells are collected by centrifugation, resuspended, and in-process QC testing performed to determine total viable cell count and cell viability. Cells may be used immediately after isolation for any methods or compositions of the disclosure.

Morphology may be evaluated immediately after isolation, at each passage, and/or prior to harvest to monitor the culture purity throughout the process. Morphology is evaluated by comparing the observed sample with visual standards for morphology examination of cell cultures. The cells may display typical CAF and/or fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. CAFs and/or fibroblasts in less confluent areas can be similarly shaped, but randomly oriented.

The presence of keratinocytes in cell cultures may also be evaluated. Keratinocytes appear round and irregularly shaped and, at higher confluence, they appear organized in a cobblestone formation. At lower confluence, keratinocytes are observable in small colonies.

In some aspect, CAFs and/or fibroblasts may be transfected and/or transduced with heterologous genes for enhanced growth and/or overcoming of the Hayflick limit. This may or may not occur subsequent to isolation and/or expansion of the cells.

### 2. Fibroblast and CAF Cell Culture

The methods of the disclosure may comprise culturing CAF and/or fibroblasts cells obtained from tissue samples, e.g., human samples. In particular aspects, the populations of CAF and/or fibroblast cells may be plated onto a substrate. In some aspects, CAFs and/or fibroblasts may be plated onto a substrate which allows for adherence of cells thereto. This may be carried out, e.g., by plating the cells in a culture plate which displays one or more substrate surfaces compatible with cell adhesion. When the said one or more substrate surfaces contact the suspension of cells (e.g., suspension in a medium) introduced into the culture system, cell adhesion between the cells and the substrate surfaces may ensue. Accordingly, the term “plating onto a substrate which allows adherence of cells thereto” refers to introducing cells into a culture system that features at least one substrate surface that is generally compatible with adherence of cells thereto, such that the plated cells can contact the substrate surface.

General principles of maintaining adherent cell cultures are well-known in the art. As appreciated by those skilled in the art, the CAF and/or fibroblast cells may be counted in order to facilitate subsequent plating of the cells at a desired density. Where the cells after plating may primarily adhere to a substrate surface present in the culture system (e.g., in a culture vessel), the plating density may be expressed as number of cells plated per mm² or cm² of the said substrate surface. In practicing the disclosure, after plating of the CAFs and/or fibroblasts, the cell suspension is left in contact with the adherent surface to allow for adherence of cells from the cell population to the substrate. In contacting CAFs and/or fibroblasts to the adherent substrate, the cells may be advantageously suspended in an environment comprising at least a medium, in the methods of the disclosure typically a liquid medium, which supports the survival and/or growth of the cells. The medium may be added to the system before, together with or after the introduction of the cells thereto. The medium may be fresh, i.e., not previously used for culturing of cells, or may comprise at least a portion which has been conditioned by prior culturing of cells therein, e.g., culturing of the cells which are being plated or antecedents thereof, or culturing of cells more distantly related to or unrelated to the cells being plated.

The medium may be a suitable culture medium as described elsewhere in this specification. In some cases, the composition of the medium may have the same features, may be the same or substantially the same as the composition of medium used in the ensuing steps of culturing the attached cells. Otherwise, the medium may be different. In some aspects, the cells from the CAF and/or fibroblast cell population or from tissue explants of the present disclosure, which may adhered to a substrate, preferably in the environment, may be subsequently cultured for at least 1, 2, 3, 4, 5, 6, 7, 8, 9, 10, 11, 12, 13, 14, 15, 16, 17, 18, 19, 20, 21 days or more. The term “culturing” is common in the art and broadly refers to maintenance and/or growth of cells and/or progeny thereof.

In some aspects, the CAF and/or fibroblast cells may be cultured for at least between about 10 days and about 40 days, for at least between about 15 days and about 35 days, for at least between about 15 days and 21 days, such as for at least about 15, 16, 17, 18, 19 or 21 days. In some aspects, the CAFs and/or fibroblasts of the disclosure may be cultured for no longer than 60 days, or no longer than 50 days, or no longer than 45 days. The tissue explants and CAFs and/or fibroblasts may be cultured in the presence of a liquid culture medium. Typically, the medium will comprise a basal medium formulation as known in the art. Many basal media formulations can be used to culture CAFs and/or fibroblasts herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art, and it is within the skill of one in the art to modify or modulate concentrations of media and/or media supplements as necessary for the CAFs and/or fibroblasts cultured. In some aspects, a culture medium formulation may be explants medium (CEM) which is composed of IMDM supplemented with 10% fetal bovine serum (FBS, Lonza), 100 U/mL penicillin G, 100 mg/mL streptomycin, and 2 mmol/L L-glutamine (Sigma-Aldrich). Other aspects may employ further basal media formulations, such as chosen from the ones above.

For use in the CAF and/or fibroblast culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., beta-mercaptocthanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zcocin. Also contemplated is supplementation of cell culture medium with mammalian plasma or sera. Plasma or sera often contain cellular factors and components that are necessary for viability and expansion. The use of suitable serum replacements is also contemplated (e.g., FBS).

The CAFs and/or fibroblasts of the disclosure, when cultured in liquid media, may show upregulation of one or more genes involved in the cell cycle (e.g., cyclins and cyclin dependent kinases) and/or in DNA replication and/or purine metabolism when compared to standard or non-modified CAFs, fibroblasts, or mesenchymal stem cells. In particular aspects, genes functionally active in cell adhesion/extracellular matrix (ECM)-receptor interaction, differentiation/development, TGF-β signaling, and TSP-1-induced apoptosis are downregulated in the CAFs and/or fibroblasts of the disclosure, when compared to standard or non-modified CAFs or mesenchymal stem cells. Specific examples of TGF-beta associated signaling genes, which are downregulated by the CAFs, include at least SMAD1, SMAD2, SMAD3, SMAD4, GITR (also known as TNF receptor superfamily member 18), programmed death-ligand 1 (PD-L1), CD5, and/or CD31.

The fibroblasts and or CAFs of the disclosure may be cultured in media supplemented with platelet lysate (PL) and/or fetal calf serum (FCS), in some cases. In some cases, the CAFs and/or fibroblasts are cultured in platelet lysate (PL) containing media. For example, 300 μL of CAFs and/or fibroblasts may be cultured in 15 mL of PL supplemented medium in a T-75 flask or other adequate tissue culture vessels. After washing away the non-adherent cells, the adherent cells are also cultured in media that has been supplemented with platelet lysate. Thrombocytes are a well characterized human product that is widely used in clinics for individuals in need of blood supplement. Thrombocytes are known to produce a wide variety of factors, e.g. platelet derived growth factor subunit b (PDGF-BB), transforming growth factor beta (TGF-β), insulin-like growth factor 1 (IGF-1), and vascular endothelial growth factor (VEGF). In some aspects, an optimized preparation of PL is used. This optimized preparation of PL is made up of pooled platelet rich plasmas (PRPs) from at least 10 donors (to equalize for differences in cytokine concentrations) with a minimal concentration of 3×10⁹ thrombocytes/mL.

According to particular aspects of the method of producing CAFs and/or fibroblasts of the disclosure, CAFs and/or fibroblasts are exposed to PL that was prepared either from pooled thrombocyto concentrates designed for human use (produced as TK5F from the blood bank at the University Clinic UKE Hamburg-Eppendorf, pooled from 5 donors) or from 7-13 pooled buffy coats after centrifugation with 200×g for 20 min. In particular cases, the PRP was aliquoted into small portions, frozen at −80° C., and thawed immediately before use to produce PL. In particular aspect, PL-containing medium is prepared freshly for each cell feeding. In a particular aspect, medium is used that contains alpha-MEM as basic medium supplemented with 5 IU Heparin/mL medium and 5% of freshly thawed PL. The method of producing CAFs and/or fibroblasts of the disclosure may use a method to prepare PL that differs from others according to the thrombocyte concentration and centrifugation forces, in some cases.

In some aspect, cells are cultured in media at about 37° C. with approximately 5% CO₂ under hypoxic conditions. In certain aspects, the hypoxic conditions are an atmosphere of 5% O₂. In some situations, hypoxic culture conditions allow CAFs and/or fibroblasts to grow faster than CAFs and/or fibroblasts cultured in non-hypoxic conditions. This allows for a reduction of days needed to grow the cells to 90-95% confluence. Generally, hypoxia can reduce the growing time by about three days. Hypoxia may also activate the cells to upregulate specific sets of genes, such as ECM genes. In some aspects, cells may be cultured in media at about 37° C. with approximately 5% CO₂ under normoxic conditions, i.e. wherein the O₂ concentration is the same as atmospheric O₂, at approximately 20.9%. In some aspects, the cells are grown to between 70 and 90% confluence. In some aspects, once this level of confluence is reached, the cells may be enzymatically detached, for example using trypsin.

In certain aspects, the population of cells that is isolated from the plate is between 85-95% CAFs and/or fibroblasts. In other aspects, the CAFs and/or fibroblasts are greater than 95%, 96%, 97%, 98%, or 99% of the isolated cell population.

In some aspects, a closed system may be used for generating and expanding the CAFs and/or fibroblasts of the disclosure. This closed system may be a device to functionally expand cells ex vivo. In one specific aspect, the closed system comprises: 1) a central expansion unit preferably constructed similarly to bioreactors with compressed (within a small unit), but extended growth surfaces; 2) media bags that can be sterilely connected to the expansion unit (e.g. by welding tubes between the unit and the bags) for cell feeding; and 3) electronic devices to operate automatically the medium exchange, gas supply and temperature. The advantages of the closed system in comparison to conventional flask tissue culture are the construction of a functionally closed system, i.e. the cell input and media bags are sterile welded to the system. This minimizes the risk of contamination with external pathogens and therefore may be highly suitable for clinical applications. Furthermore, this system can be constructed in a compressed form with consistently smaller cell culture volumes but preserved growth area. The smaller volumes allow the cells to interact more directly with each other which creates a culture environment that is more comparable to the in vivo situation of the bone marrow niche. Also, the closed system saves costs for the media and the whole expansion process.

The construction of the closed system may involve two sides: the cells are grown inside of multiple fibers with a small medium volume. In some aspects, the culture media comprises growth factors for growth stimulation, and medium without expensive supplements is passed outside the fibers. The fibers are designed to comprise nanopores for a constant removal of potentially growth-inhibiting metabolites while important growth-promoting factors are retained in the growth compartment.

In certain aspects, a closed system may be used in conjunction with a medium for expansion of CAFs and/or fibroblasts that does not contain any animal proteins, e.g., fetal calf serum (FCS). FCS has been connected with adverse effects after in vivo application of FCS-expanded cells, e.g., formation of anti-FCS antibodies, anaphylactic or arthus-like immune reactions or arrhythmias after cellular cardioplasty. FCS may introduce unwanted animal xenogeneic antigens, viral, prion and zoonose contaminations into cell preparations making new alternatives necessary.

### 3. Fibroblast and CAF Markers

In some aspects, CAF and/or fibroblasts cells of the present disclosure are identified and characterized by their expression of specific marker genes, such as cell-surface markers. Detection and isolation of these cells can be achieved, e.g., through flow cytometry, FACS, immunohistochemistry, immunofluorescence, imaging, ELISA, and/or magnetic bead separation. Reverse-transcription polymerase chain reaction (RT-PCR) can also be used to monitor changes in gene expression.

In certain aspects, markers used to identify and/or characterize a fibroblast and/or CAF comprise c-Kit (also known as CD117), has related family bHLH transcription factor with YRPW motif 1 (Heyl), α-smooth muscle actin (SMA), Vimentin (including intracellular or extracellular vimentin), Cyclin D2, Snail, E-cadherin, NK2 homeobox 5 (Nkx2.5), GATA binding protein 4 (GATA4), cluster of differentiation (CD) 105, CD271, CD90, CD29, CD73, CD44, CD10, CD13, CD44, Wilms' tumor 1 (Wt1), CXC motif chemokine receptor 4 (CXCR-4), fibroblast growth factor 1 (FGF-1) receptor, stage specific embryonic antigen 3 (SSEA-3), tumor necrosis factor alpha (TNF-α) receptor-1, toll like receptor 4 (TLR4), receptor for acetylated end products (RAGE), hepatocyte growth factor (HGF), express octamer-binding transcription factor 4 (Oct-4), CD-34, Krüppel-like factor 4 (KLF-4), Nanog, Sox-2, Rex-1 (also known as zinc finger protein 42), growth differentiation factor 3 (GDF-3), Stella (also known as developmental pluripotency-associated 3), or possess enhanced expression of GDF-11, and a combination thereof. In certain aspects, markers used to identify and/or characterize CAFs comprise FAP, POSTN, PDGFRα/β, FSP-1, CD90, Palladin, OPN, AEBP1, Twist, TNC, Gallectin1, CD10 and GPR77, Cav-1, PDPN, CD200, or a combination thereof. Methods and markers to characterize CAFs are described in Zhao Z, Li T, Yuan Y, Zhu Y. What is new in cancer-associated fibroblast biomarkers? Cell Commun Signal. 2023 May 4; 21 (1): 96, which is incorporated in its entirety herein by reference.

In specific aspects, fibroblasts or CAFs and mesenchymal stem cells (MSCs) are distinguishable from each other because MSCs do not express octamer-binding transcription factor 4 (Oct-4), CD-34, Krüppel-like factor 4 (KLF-4), Nanog, Sox-2, Rex-1 (also known as zinc finger protein 42), growth differentiation factor 3 (GDF-3), Stella (also known as developmental pluripotency-associated 3), or possess enhanced expression of GDF-11.

In specific aspects, CAF and fibroblasts are distinguishable from each other because CAFs express FAP, PDGFR, Vimentin, PDPN, CD70, CD49c, CD10/GRP77 and MHCII/CD74, TIMP-1, SPARC, COL1A2, COL3A1, COL1A1, or a combination thereof. Methods and markers used to differentiate fibroblasts and CAFs are described in Dwivedi N, Shukla N, Prathima K M, Das M, Dhar S K. Novel CAF-identifiers via transcriptomic and protein level analysis in HNSC patients. Sci Rep. 2023 Aug. 25; 13 (1): 13899, which is incorporated in its entirety herein by reference.

In specific embodiments, CAFs of the disclosure may possess the ability to create a barrier against immune cells (e.g., CD8⁺ T cells) and/or immune responses (e.g., anti-tumor immune responses). In some embodiments, CAFs of the disclosure may possess the ability to kill immune cells (e.g., CD8⁺ T cells). In some embodiments, CAFs of the disclosure may possess the ability to kill immune cells (e.g., CD8⁺ T cells) via PD-L2 and/or FASL engagement. In some embodiments, CAFs of the disclosure may possess the ability to block proteins (e.g., antibodies) from reaching encapsulated cells. Characteristics possess by CAFs are described in Zhang H, Yue X, Chen Z, Liu C, Wu W, Zhang N, Liu Z, Yang L, Jiang Q, Cheng Q, Luo P, Liu G. Define cancer-associated fibroblasts (CAFs) in the tumor microenvironment: new opportunities in cancer immunotherapy and advances in clinical trials. Mol Cancer. 2023 Oct. 2; 22 (1): 159, which is incorporated in its entirety herein by reference.

### 4. Activated Fibroblasts and CAFs

In some aspects, CAFs and/or fibroblasts may be activated prior to, or during, use for methods disclosed herein. Aspects of the disclosure include the use of CAFs, fibroblasts, fibroblasts activated with one or more agents, and/or CAFs activated with one or more agents. In particular aspects, CAFs and/or fibroblasts or other cells encompassed herein become activated following exposure to one or more conditions and/or agents. Specific agents that may modify the CAFs and/or fibroblasts include one or more of nucleic acids, cytokines, chemokines, growth factors, and/or exosomes prior to and/or during their use. The CAF and/or fibroblast cells may be activated, such as having activated or engineered surface markers, nucleic acid modification, and/or expression or secretion of one or more chemokines, one or more cytokines, exosomes, extracellular matrix components, and/or one or more growth factors. In specific aspects, the CAFs and/or fibroblasts are activated by exposing them to other cells (e.g., platelets, neutrophils, monocytes, macrophages (e.g., M1, M2, M2a), dendritic cells (e.g., Langerhans cells), T cells (e.g., αβ T cells, γδ T cells, T helper cells, regulatory T cells, killer T cells), endothelial cells, pericytes, hematopoietic progenitor cells, epidermal cells, epidermal stem cells, smooth muscle cells, lymphocytes, mast cells, NK cells, adipose stromal cells, immune cells, keratinocytes, epithelial cells, other fibroblasts, or a combination thereof), cancer cells, endocrine cells (e.g., beta cells, alpha cells, delta cells, epsilon cells, and/or pancreatic polypeptide cells). The cells may be activated by one or more chemical agents, RNA, micro RNA, RNAi, DNA, viral nucleic acid, and/or exosomes. In specific aspects, the cytokine is selected from the group consisting of IFN-gamma, TNF-alpha, interleukin (IL)-1, IL-6, IL-7, IL-8, IL-12, IL-15, IL-17, IL-33, and a combination thereof. In specific aspects, the growth factor is selected from the group consisting of FGF-1, VEGF, and a combination thereof. In specific aspects, the cells are activated following exposure to human platelet-rich plasma, platelet lysate, umbilical cord blood serum, autologous serum, human serum, serum replacement, or a combination thereof. In specific aspects, the cells are activated following exposure to hypoxia. The hypoxia may be 0.1%-10%, 0.1%-5%, 0.1%-2.5%, or 0.1%-1% oxygen, for example. In specific aspects, the hypoxia occurs for a period of time that is at least or no more than between about 30 minutes to about 3 days, although in some cases, it may be less than 30 minutes or greater than 3 days. The CAFs may be exposed to one or more agents prior to and/or during exposure to hypoxia, carbon monoxide, or a combination thereof. In some aspects, the CAFs and/or fibroblasts may be activated by exposure to oxidative stress. In some aspects, the CAFs and/or fibroblasts may be activated by exposure to growth factors produced by cancer cells, such as TGF-β, epidermal growth factor (EGF), fibroblast growth factor type 2 (FGF2), PDGF, Activin A, Nodal, or one or more BRAF inhibitors (e.g., Vemurafenib, dabrafenib, encorafenib, or a combination thereof), or a combination thereof. In some aspects, CAFs and/or fibroblasts may be activated to secrete ECM by cytokines, chemokines, growth factors, transcription factors, and/or nucleic acids. In some aspects, CAFs and/or fibroblasts are activated by exposure to CAF and/or fibroblast derived materials, such as exosomes, lysate, extracellular vesicles, membrane fragments, or a combination thereof.

### 5. Fibroblast and CAF Organoids

Aspects of the disclosure may utilize organoids. In specific aspects, the organoids are produced such that they may (1) initiate or maintain immune modulatory activity, (2) initiate or maintain extended time release of activated or inactivated therapeutic factors (e.g., insulin), and/or (3) initiate or maintain extended time release migration and proliferation to a targeted tissue or organ for any purpose, including for repair, regeneration and/or reversal of involution. The cell structure of the organoid, as opposed to a plurality of single cells, allows for enhanced cell contact, and in some aspects of the invention allow for protection of cells in the inner core of the organoid from the effects of the immune system. Methods of production or use of organoids are described in PCT/US2023/076365, which is incorporated herein by reference in its entirety.

As an initial step of the methods, or soon after an initial step of the methods, CAFs and/or fibroblasts cells may be manipulated to form organoids (including spheroids) under appropriate suspension culture conditions. In some aspects for CAF and/or fibroblast spheroid formation, a U/V bottom type ultra-low attachment culture plate or hanging drop method can be used, according to different conditions. For example, cell organoids of different sizes may be generated according to the density of CAFs and/or fibroblasts in the medium. In some cases, surface tension on a substrate is manipulated to enhance formation of the organoids. In a specific case, a surface is treated with a hydrophobic material except for a hydrophilic area upon which individual cells or clumps of cells are placed to facilitate production of the organoid. In some cases, spheroids can be formed by forming a core-shell structure within a pipette tip (e.g., (1) making the core spheroid (e.g., using insulin producing cells) in a single pipette tip and culturing for 48 hours; (2) creating a second layer on the spheroids with cells (e.g., CAFs and/or fibroblasts) in the same pipette tip and culturing for 48 hours, repeating as desired to add additional layers of cells and/or cell types to the spheroid; and (3) after the cells spheroids form in the pipette tip for 96 hours, collecting the cell spheroids from the pipette tip by ejecting the liquid containing the spheroid from inside the pipette tip, removing the liquid matrix using centrifugation or filtration, and collecting the cell spheroids). In some aspects, a spheroid can be generated by plating cells in a microwell or microcavity. In some aspects, cells plated in a microwell or microcavity are centrifuged to promote spheroid formation. It is also possible to directly use ultra-low attachment culture plates or glass culture flasks to spread CAFs and/or fibroblasts into vessels or culture dishes at high density with low-speed stirring to form clumps of different sizes spontaneously. CAFs and/or fibroblasts may be used to form any part of a spheroid, such as an inner core or one of more outer shell layers. CAFs and/or fibroblasts may be used with other cell types, e.g., insulin producing cells, to form organoids.

In particular aspects, the size of the spheroids can range from 50 microns to 500 microns, although small and larger sizes may also be used. In particular aspects, the sphere size can be between about 50-500, 50-400, 50-300, 50-200, 50-100, 100-500, 100-400, 100-300, 100-200, 200-500, 200-400, 200-300, 300-500, 300-400, or 400-500 microns, including any value or range derivable therein. The sphere size may be about 50, 75, 100, 125, 150, 175, 200, 225, 250, 275, 300, 325, 350, 375, 400, 425, 450, 475, or 500 microns, including any value or range derivable therein. In specific cases, the size of the organoid is generally controlled by the duration of time of culture and the culture media, optionally among other culture parameters.

In particular aspects, the organoids comprise a particular range of cell numbers. In specific cases, the number of cells in the organoid are generally controlled by the duration of time of culture and the culture media, optionally among other culture parameters. In specific aspects, the organoids comprise at least about or no more than about 10³, 10⁴, 10⁵, 10⁶, 10⁷, 10⁸, 10⁹, or 10¹⁰ cells per organoid, including any value or range derivable therein. In some aspects, a spheroid comprises an inner core comprising about 1 to 100,000 cells. In some aspects, the inner core of a spheroid comprises, consists of, or consists essentially of insulin producing cells and/or one or more outer shell layers of CAFs and/or fibroblasts.

In some aspects, spheroids are generated upon culture in an incubator at about 37° C., such as for about 24-48 hours. The incubator may or may not be set at about 5% carbon dioxide and greater than about 80% humidity. Cell clumps can also form spontaneously at 20-37° C., although other conditions may be utilized therewith, in some cases.

In particular aspects, for formation, the spheroids may be cultured for 24-48, 24-44, 24-40, 24-36, 24-30, 24-28, 28-48, 28-44, 28-40, 28-36, 28-30, 30-48, 30-44, 30-40, 30-36, 36-48, 36-44, 36-40, 40-48, 40-44, or 44-48 hours, including any value or range derivable therein, in an incubator of suitable conditions. In specific aspects, the incubator may be set at a particular temperature, such as at about 20, 21, 22, 23, 24, 25, 26, 27, 28, 29, 30, 31, 32, 33, 34, 35, 36, or 37° C., including any value or range derivable therein. A range of temperatures includes about 20-37, 20-35, 20-30, 20-27, 20-25, 20-22, 22-37, 22-35, 22-33, 22-30, 22-27, 22-25, 25-37, 25-35, 25-33, 25-30, 25-27, 27-37, 27-35, 27-33, 27-30, 30-37, 30-35, 30-33, 33-37, 33-35, or 35-37° C., including any value or range derivable therein.

In specific aspects, the incubator may utilize a certain percentage of a gas, such as carbon dioxide, oxygen, and/or nitrogen. The carbon dioxide may be at 3, 4, 5, 6, 7, or 8%. Ranges of carbon dioxide include about 3-8, 3-7, 3-6, 3-5, 3-4, 4-8, 4-7, 4-6, 4-5, 5-8, 5-7, 5-6, 6-8, 6-7, or 7-8%, including any value or range derivable therein. In specific aspects, the incubator may be set at a particular percentage of humidity, such as about 65, 70, 75, 80, 85, or 90% humidity, including any value or range derivable therein. The oxygen in the incubator may be 0.5-25% oxygen, including 0.5-25, 0.5-20, 0.5-15, 0.5-10, 0.5-5, 0.5-1, 1-25, 1-20, 1-15, 1-10, 1-5, 5-25, 5-20, 5-15, 5-10, 10-25, 10-20, 10-15, 15-25, 15-20, or 20-25%, including any value or range derivable therein. The oxygen in the incubator may be 0.5, 1, 2, 3, 4, 5, 6, 7, 8, 9, 10, 11, 12, 13, 14, 15, 16, 17, 18, 19, 20, 21, 22, 23, 24, or 25%, including any value or range derivable therein. The nitrogen in the incubator may be 1-75% nitrogen, including 1-75, 1-70, 1-60, 1-50, 1-40, 1-30, 1-20, 1-10, 1-5, 5-75, 5-70, 5-60, 5-50, 5-40, 5-30, 5-20, 5-10, 10-75, 10-70, 10-60, 10-50, 10-40, 10-30, 10-20, 20-75, 20-70, 20-60, 20-50, 20-40, 20-30, 30-75, 30-70, 30-60, 30-50, 30-40, 40-75, 40-70, 40-60, 40-50, 50-75, 50-70, 50-60, 60-75, 60-70, or 70-75% nitrogen, including any value or range derivable therein. The nitrogen in the incubator may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75% nitrogen, including any value or range derivable therein.

A range of humidities may be utilized, such as about 65-90, 65-85, 65-80, 65-75, 65-70, 70-90, 70-85, 70-80, 70-75, 75-90, 75-85, 75-80, 80-90, 80-85, or 85-90%, including any value or range derivable therein. Other incubator conditions and culture durations may be present or required.

In various aspects, the size of the produced spheroid is able to be controlled at least in part. The desired size may be dictated for one or more purposes, such as a desired size for production purposes, a desired size for transportation purposes, and/or a desired size for a research and/or therapeutic applications. In some aspects, a plurality of organoids to be utilized comprise substantially the same size. In some aspects, a first plurality of organoids may be combined with a second plurality of organoids of a different size before transportation and/or research and/or therapeutic application; in such cases, the first and second pluralities may have been produced under different conditions, such as different culture parameters, including different durations of culture time. The produced size of the organoids may be dictated by adjusting the concentration of cells used to produce spheroids. Such adjusting of the concentration may or may not occur by dilution; by the selection of the base media; by the selection of one or more additives to the media (such as sugar, serum, non-essential amino acids, L-glutamine, a combination thereof, etc.); by selection of the amount of carbon dioxide in the incubator; by the temperature in the incubator; by the duration of time of production of the spheroid; using an outside source of a gas or gas mixtures to feed the incubator; or a combination thereof. The concentration may be increased for certain purposes and decreased for certain purposes. The adjustment of the concentration may increase the size of the organoid by an approximate certain fold level, such as about 2-fold, 5-fold, 10-fold, 100-fold, 1000-fold, 10,000-fold, etc., including any value or range derivable therein. The adjustment of the concentration may decrease the size of the organoid by an approximate certain fold level, such as about 2-fold, 5-fold, 10-fold, 100-fold, 1000-fold, 10,000-fold, etc., including any value or range derivable therein.

In some aspects, the size of the organoids can be modified to accommodate varying clinical needs, such as adjusting the concentration of the cells and/or adjusting the duration of time of growth of the organoid. For example, in aspects wherein a tissue is in need of repair, regeneration and/or reversal of involution, the size of the organoid may be particularly produced, depending on the tissue site in need of the organoids. In some aspects, the organoid is utilized as a therapeutic delivery agent wherein the therapeutic(s) delivered by the organoid is the inner core cells and/or the outer shell cells. In such cases, the organoid may act as an extended time release mechanism that allows for the release of the therapeutic(s) over a desired period of time. The desired period of time may be when there is a detectable improvement of symptoms (as detectable by appropriate assaying means). The desired period of time may be when there is complete healing (as detectable by appropriate assaying means). In some cases, the desired period of time may be the time to be able to initiate or maintain a phenotype. In some cases, the desired period of time may be the time to produce sufficient amounts of a therapeutic agent to produce a therapeutic effect in an individual. In some cases, the desired period of time may be the time to produce sufficient amounts of insulin to manage the sugar blood levels of an individual.

In some aspects, the organoids following production are stored under suitable conditions. In at least specific cases, the temperature of the storage environment is about 2° C. to about 37° C. In specific aspects, the storage environment is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37° C., including any value or range derivable therein. The storage temperature may be in range of suitable temperatures, including at least about 2-37, 2-35, 2-30, 2-25, 2-20, 2-15, 2-10, 2-5, 5-37, 5-35, 5-30, 5-25, 5-20, 5-15, 5-10, 10-37, 10-35, 10-30, 10-25, 10-20, 20-37, 20-35, 20-30, 20-25, 25-37, 25-35, 25-30, 30-37, 30-35, or 35-37° C., including any value or range derivable therein.

Once the organoids are generated, they may be stored and/or they may be transported to an intended destination. The organoids may be suspended in a liquid base medium that includes one or more tackifiers to produce a liquid storage matrix. The liquid portion of the liquid storage matrix may comprise any base cell culture medium compatible with CAF, fibroblast and/or other cells comprising the organoid (e.g., insulin producing cells), such as one containing basic nutrients and an acid-base balance system; the components may comprise low-glucose medium, such as DMEM low-glucose medium and/or another medium containing 1 mM to 20 mM (e.g., 5 mM) glucose; anywhere from about 0 to 20% human serum; about 0 to 5% non-essential amino acids; and/or about 0 to 5% L-glutamine. The tackifier may be a food-grade additive that can increase the viscosity of the liquid matrix, such as Methylcellulose or any other agent capable of increasing viscosity, and may be at a specific concentration of anywhere from about 0 to 5%. The base medium may contain other materials and/or conditions to facilitate culturing, such as growth factors, sugars, and/or amino acids.

In aspects wherein the liquid storage matrix for storage and/or transportation may comprise serum, including human serum, the amount may be about 0-20, 0-15, 0-10, 0-5, 1-20, 1-15, 1-10, 1-5, 5-20, 5-15, 5-10, 10-20, 10-15, or 15-20% human serum, including any value or range derivable therein. The amount of serum may be about 0, 1, 2, 3, 4, 5, 6, 7, 8, 9, 10, 11, 12, 13, 14, 15, 16, 17, 18, 19, or 20%, including any value or range derivable therein.

In aspects wherein the media for storage and/or transportation may comprise non-essential amino acids, the amount may be about 0-5, 0-4, 0-3, 0-2, 0-1, 1-5, 1-4, 1-3, 1-2, 2-5, 2-4, 2-3, 3-5, 3-4, or 4-5% non-essential amino acids, including any value or range derivable therein. The amount of non-essential amino acids may be about 0, 1, 2, 3, 4, or 5% non-essential amino acids, including any value or range derivable therein. The non-essential amino acids may include glycine, L-alanine, L-asparagine monohydrate, L-aspartic acid, L-glutamic acid, L-proline, L-serine, L-histidine, Isoleucine, L-lysine hydrochloride, L-serine, L-tryptophan, and/or L-valine.

In aspects wherein the liquid storage matrix for storage and/or transportation may comprise about 0 to 5% L-glutamine, the amount may be 0-5, 0-4, 0-3, 0-2, 0-1, 1-5, 1-4, 1-3, 1-2, 2-5, 2-4, 2-3, 3-5, 3-4, or 4-5% L-glutamine, including any value or range derivable therein. The amount of L-glutamine may be about 0, 1, 2, 3, 4, or 5% L-glutamine, including any value or range derivable therein.

In aspects wherein the liquid storage matrix for storage and/or transportation may comprise one or more tackifiers, the amount may be 0-5, 0-4, 0-3, 0-2, 0-1, 1-5, 1-4, 1-3, 1-2, 2-5, 2-4, 2-3, 3-5, 3-4, or 4-5% tackifiers, including any value or range derivable therein. In aspects wherein the media for storage and/or transportation may comprise one or more tackifiers, the amount of tackifier may be about 0, 1, 2, 3, 4, or 5%, including any value or range derivable therein. In cases wherein two or more tackifiers are used, their sum amount may or may not be about 0-5, 0-4, 0-3, 0-2, 0-1, 1-5, 1-4, 1-3, 1-2, 2-5, 2-4, 2-3, 3-5, 3-4, or 4-5%, including about 0, 1, 2, 3, 4, or 5%, including any value or range derivable therein.

In some aspects, upon generation the spheroids are housed in a suitable container. The range of spheroids per container may vary according to need, but in specific cases the range is 1-10 million cells per milliliter of preservation liquid matrix. In specific aspects, the packaging and preservation of the spheroids are in a sealed container, including a sterile one. In specific aspects, the container is a plastic container, such as one made from chemically inert materials including polyethylene, polypropylene, and melamine. The container may also be constructed of other materials that would not adversely impact the cells, such as glass, coated metal, or other polymers such as polystyrene, branched polymer hydrogels, and cycle-olefin polymers.

Aspects of the present disclosure allow for the spheroids to be transported under ambient conditions, and this may be before or after storage. In specific aspects, the spheroids are transported in a temperature range from 2-35° C. The spheroids may be transported in a temperature range of 2-35, 2-30, 2-25, 2-20, 2-15, 2-10, 2-5, 5-35, 5-30, 5-25, 5-20, 5-15, 5-10, 10-30, 10-25, 10-20, 10-15, 15-35, 15-30, 15-25, 15-20, 20-35, 20-30, 20-25, 25-35, 25-30, or 30-35° C., including any value or range derivable therein. The spheroids may be transported at 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35° C., including any value or range derivable therein. In specific aspects, the temperature for transport is not more than about 38° C. and/or is not less than 0° C., although in other aspects the spheroids are transported cryogenically.

In specific aspects, the cell container is not subject to intense energy or light waves, including intense light irradiation, X rays, such as is used at airports, or any intense light source during at least part if not all of transportation. In specific cases, the cell container and/or the incubator during preparation is not subject to light during at least part if not all of transportation.

The produced organoids may be transported and/or used within about 0-50 days following production. In some cases, the organoids are transported and/or used on the same day as production, within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, to 50 days, including any value or range derivable therein. In some aspects, the organoids are transported and/or used within 1-50, 1-40, 1-30, 1-25, 1-20, 1-10, 1-5, 5-50, 5-40, 5-30, 5-25, 5-20, 5-10, 10-50, 10-40, 10-30, 10-25, 10-20, 20-50, 20-40, 20-30, 20-25, 25-50, 25-40, 25-30, 30-50, 30-40, or 40-50 days, including any value or range derivable therein.

In some aspects, the size of the organoids can be modified to accommodate varying clinical needs, such as adjusting the concentration of the cells and/or adjusting the duration of time of growth of the organoid. For example, larger organoids may be able to produce more therapeutic agents (e.g., insulin) than smaller organoids. However, a specific size of organoids may be desired for injection from a needle or for implantation via other means. In some aspects, specific size of organoid is desired to be able to be infused to a specific site in which the vein/artery size is considered.

In particular aspects, one can modulate the size of the fibroblast spheroids to adjust the extent and duration of the extended-release function of therapeutic agents by cells of the organoid in vivo. Such modulation may encompass producing larger-sized organoids for longer extended release of the therapeutic agents. In some aspects, the size of the organoids may contribute to the effectiveness of the therapy. For example, smaller organoids may be preferable to better protect the inner core cells from the immune system. In other cases, a larger shell size may be desired to provide extra protection to the inner core cells.

In aspects of the disclosure, one may utilize the organoids as a method of “extended time release” of therapeutic agents produced by the organoid. Such agents may be used for any suitable purpose, including for the purpose of immune modulation, chronic disease treatment, tissue repair, tissue regeneration, targeting of specific tissue for migration, and/or proliferation for subsequent growth, and/or differentiation.

In specific aspects, the therapeutic agents released from the organoids may be released slowly over a period of time from the spheroid to elicit continuous and sustained therapeutic response, such as modulation of sugar in the blood of an individual. In specific aspects, the organoids may react in response to changes in the environment. For example, when the concentration of sugar in the blood increases, the organoids may release more insulin, and when the concentration reaches appropriate levels, the organoid may cease, or decrease, production of insulin.

In particular aspects, the organoids are administered to an individual in need thereof by any suitable administration, including one that is selected for the application being treated. Such administration may be local or systemic and may include injection, infusion, spray, and/or inclusion in a 3D matrix. In specific aspects, the organoids may be comprised in a carrier for implantation into a specific tissue or organs, e.g., the pancreas. In specific aspects, there is one administration to the individual or multiple administrations. In cases wherein there are multiple administrations, the duration in time between the administrations may be within 1, 2, 3, 4, 5, 6, 7, 8, 9, 10, 11, or 12 hours, or within 1, 2, 3, 4, 5, 6, or 7 days, or within 1, 2, 3, or 4 weeks, or within 1, 2, 3, 4, 5, 6, 7, 8, 9, 10, 11, or 12 months, or within 1, 2, 3, 4, 5, or more years between administrations, including any value or range derivable therein. In specific aspects, 1, 2, 3, 4, 5, 6, 7, 8, 9, 10 or more administrations may occur within a 24 hour period.

### B. Insulin Producing Cells

In some aspects, the encapsulated cells (e.g., the inner core cells of an organoid) are insulin producing cells. In some aspects, the encapsulated cells are pancreatic islet cells (e.g., pancreatic beta cells). In some aspects, the encapsulated cells are cells engineered to secrete a therapeutic agent, such as, but not limited to, insulin.

### 1. Pancreatic Islet Cells

In order to assist the practitioner of this disclosure, methods of isolating pancreatic islet cells for use in methods and compositions of the disclosure are provided and incorporated by reference. In one aspect, the methodology described in U.S. Patent Application US2006/0182722 are provided: Pancreases can be obtained from male or female donors via techniques developed for combined liver and pancreaticoduodenal procurement (Marsh et al., Surg. Gynecol. Obstet. 1989; 168:254-258). Pancreatic beta cells may be derived from cadavers. Donors typically range in age from 15 to 50 years old. General exclusion criteria include, for example, systemic bacterial infections, viruses such as human immunodeficiency virus (HIV), human T-cell lymphotrophic virus (HTLV), hepatitis B virus, or hepatitis C virus (HCV), a history of diabetes, extracranial tumors, and risk factors for AIDS. Donor pancreases can be preserved using the two-layer pancreas preservation method, which improves pancreatic tissue adenosine triphosphate (ATP) content, increases the yield of islets isolated from a stored pancreas, allows use of marginal donor pancreases for islet isolation and transplantation, improves the islet isolation success rate, and preserves the integrity of the isolated islets (e.g., such that isolated islets can reverse diabetes). In general, cold University of Wisconsin (UW) Solution (ViaSpan®, DuPont Pharma, Wilmington, Del.) (see U.S. Pat. Nos. 4,798,824 and 4,879,283, incorporated herein by reference) or modified UW solution can be poured on top of an equal volume of cold perfluorodecalin (FluoroMed, L. P., Round Rock, Tex.). Typically, the two-layer preservation method is performed in an organ shipping container, which has, for example, a removable lid with a stainless steel mesh plate attached thereto, and inlet and outlet ports. See, for example, the organ shipping container of U.S. Pat. No. 6,490,880, incorporated herein by reference. Two layers are formed after adding ViaSpan® or modified-UW solution to the perfluorodecalin as the specific gravity of perfluorodecalin is greater than ViaSpan®. and modified-UW solution. Modified UW solution includes 0.35 to 0.45 g/L potassium hydroxide, 3.00 to 4.00 g/L monosodium phosphate monohydrate, 0.05 to 1.00 g/L calcium chloride dihydrate, 1.10 to 1.30 g/L magnesium sulfate heptahydrate, 33.00 to 38.00 g/L lactobionic acid, 4.00 to 5.00 g/L L-histidine, 15.00 to 20.00 g/L raffinose, 4.00 to 5.00 g/L sodium hydroxide, 15.00 to 25.00 g/L penta starch, 1.00 to 1.50 g/L adenosine, and 0.75 to 1.50 g/L glutathione. In particular, the modified UW solution can include 0.39 g/L potassium hydroxide, 3.45 g/L monosodium phosphate monohydrate, 0.074 g/L calcium chloride dihydrate, 1.23 g/L magnesium sulfate heptahydrate, 35.83 g/L lactobionic acid, 4.66 g/L L-histidine, 17.84 g/L raffinose, 4.60 g/L sodium hydroxide, 20.00 g/L penta starch, 1.34 g/L adenosine, and 0.92 g/L glutathione. Typically, the perfluorodecalin is oxygenated for 30-70 minutes (e.g., 40-60 minutes). For example, medical grade oxygen can be filtered through a 0.2 mm filter (Gelman Sciences, Ann Arbor, Mich.) in through the inlet port of the shipping container at a rate of 2.5 L/min. In some embodiments, the cold storage time of the donor pancreas is less than 12 hours (e.g., less than 10, 8, 6, 4, or 2 hours). Upon receipt of a donor pancreas, integrity of the shipping container can be verified by visual inspection. The pancreas can be removed and rinsed with cold transport solution containing 8.00 to 10.00 g/L mannitol, 3.00 to 6.00 g/L L-histidine, 18.00 to 21.00 g/L gluconic acid, 0.50 to 2.00 g/L potassium hydroxide, 0.01 to 0.05 g/L calcium chloride, 0.50 to 2.00 g/L magnesium sulfate, 0.40 to 0.80 g/L nicotinamide, 0.30 to 0.70 g/L pyruvate, and 1.50 to 3.50 g/L potassium phosphate monobasic. For example cold transport solution can include 8.50 to 9.50 g/L (e.g., 9.11 g/L) D-mannitol, 4.00 to 5.00 g/L (e.g., 4.67 g/L) L-histidine, 18.50 to 20.50 g/L (e.g., 19.63 g/L) D-gluconic acid sodium salt, 0.80 to 1.40 g/L (e.g., 1.12 g/L) potassium hydroxide, 0.025 to 0.045 g/L (e.g., 0.037 g/L) calcium chloride dihydrate, 1.00 to 1.50 g/L (e.g., 1.23 g/L) magnesium sulfate heptahydrate, 0.55 to 0.65 g/L (e.g., 0.61 g/L) nicotinamide, 0.50 to 0.60 g/L (e.g., 0.55 g/L) sodium pyruvate, and 2.50 to 3.25 g/L (e.g., 2.72 g/L) potassium phosphate monobasic.

Islets can be isolated from the donor pancreas using an automated method of pancreatic tissue dissociation. Sec, for example, Ricordi et al., Diabetes 1988; 37:413-420, incorporated herein by reference. This method includes the general steps of 1) dissection; 2) distension; 3) dissociation; and 4) collection. Dissection of the pancreas can include removing extraneous fat (while retaining some fat to minimize leaking during distension), and non-pancreatic tissue. Typically, about 80% to about 95% of the fat is removed. The dissected pancreas can be incubated in a topical antibiotic solution containing, for example, gentamicin (Elkins-Sinn, Inc.), Cefazolin (SmithKline Beecham Pharmaceutical), and amphotericin-B (Apothecon®) in cold transport solution, then can be serially rinsed in phenol red-free Hanks' Balanced Salt Solution (Mediatech, Inc., Herndon, Va.). The pancreas can be divided at the neck into the “body and tail” and “head”, and the following steps performed on each part. In general, the pancreatic duct can be cannulated with an angiocatheter (16-20 gauge), and the pancreas perfused under controlled conditions, including an initial pressure of 80 mmHg followed by an increase in pressure to 180 mmHg for the remainder of the distension procedure. Phase I solution can be used to perfuse the pancreas. Phase I solution includes 5.00 to 6.00 g/L mannitol, 0.50 to 0.70 g/L sodium hydroxide, 5.00 to 7.00 g/L sodium chloride, 0.25 to 0.40 g/L potassium hydroxide, 0.05 to 0.15 g/L calcium chloride, 0.15 to 0.25 g/L magnesium sulfate, and 3.00 to 4.00 g/L sodium phosphate monobasic. For example, Phase I solution can include 5.47 g/L D-mannitol, 0.60 g/L sodium hydroxide, 6.14 g/L sodium chloride, 0.33 g/L potassium hydroxide, 0.11 g/L calcium chloride dihydrate, 0.20 g/L magnesium sulfate heptahydrate, and 3.45 g/L sodium phosphate monobasic. Typically, the Phase I solution contains 1,000 to 3,600 Wunsch units (collagenase activity) or 28,000 to 128,500 cascinase units (proteolytic activity) of collagenase. For example, the Phase I solution can include 1500 to 3000 (e.g., 1,562 to 2,954 or 2,082 to 2,363) Wunsch units, or 42,000 to 108,000 (e.g., 42,328 to 107,064 or 56,437 to 85,651) cascinase units of collagenase. A suitable collagenase includes Liberase™HI (Roche Molecular Biochemicals, Indianapolis, Ind.), which has been specifically formulated for human islet isolation procedures. Sec Linetsky et al., Diabetes 1997; 46:1120-1123, incorporated herein by reference. In some embodiments, powdered Liberase™HI is reconstituted at least 20 minutes before, but less than 2 hours before, in addition to the Phase I solution. The Phase I solution also can include a protease inhibitor (e.g., a trypsin inhibitor such as 4-(2-aminoethyl)-benzenesulfonyl fluoride hydrochloride (Pefabloc® SC PLUS), TLCK (1-Chloro-3-tosylamido-7-amino-2-heptanone HCl), or trypsin inhibitor from soybean). For example, the Phase I solution can include 0.05 to 0.15 mg/mL of Pefabloc® SC PLUS, which specifically inhibits endogenous proteases and decreases auto-digestion. The Phase I solution also can include 8 to 12 units/mL of heparin (e.g., Monoparin®, Accurate Chemical and Scientific Corporation). For example, the Phase I solution can include 10 units/mL of heparin. In some aspects, the Phase I solution contains 1,000 to 3,600 Wunsch units of collagenase, 0.05 to 0.15 mg/mL of a trypsin inhibitor, and 10 units/mL of heparin. After a sufficient period of time of cold perfusion, e.g., 8-20 minutes, the distended pancreas can be further trimmed of remaining capsule and placed into a dissociation chamber (e.g., a sterile stainless steel chamber (Wahoff et al., Ann. Surg. 1995; 222:562-579, incorporated herein by reference), also known as a Ricordi chamber). Collagenase that “leaked” from the distended pancreas can be added to the chamber. Typically, the Ricordi chamber is in a circulation system that includes a heat exchange coil (e.g., a stainless steel coil), a pump, a temperature monitor and sensor, a loading flask, a fluid collection flask, a sample collecting flask, and tubes for fluidly connecting components. Flow direction can be controlled using, for example, valves or clamps. The heat exchange coil can be placed in a water bath. In one aspect of a circulation system that contains a Ricordi chamber, there are a stainless steel coil for heat exchange, six (6) tubes with small diameter (Master Flex tubing, size 16), four (4) tubes with large diameter (Master Flex tubing, size 17), steel 3-way stopcock for sampling, four (4) plastic clamps, 250 ml conical tube, tri-pour graduated disposable beaker, 1000 mL, bell-shaped plastic cover, two (2) T-connectors, (1) T-connector with Luer lock port, and one (1) Y-connector, 18 inch steel ring stand with two arms, Ismatec pump, Mon-a-therm temperature monitor and sensor, and water bath. The system can be filled with Phase I solution and air evacuated to begin the digestion phase. In particular, Phase I solution can be allowed to flow from the loading flask through the pump, heat exchange coil, and Ricordi chamber to the fluid collecting flask. After 10% to 30% of the volume of Phase I solution reaches the fluid collecting flask, the flow of the system can be adjusted such that the Phase I solution is recirculated through the system, i.e., the Phase I solution flows from the fluid collecting flask to the chamber and from the chamber to the fluid collecting flask. The chamber can be agitated while the fluid is being recirculated to aid tissue dissociation. Temperature of the fluid can be maintained at 25° C. to 37° C. The collection phase can begin once there is an increase in the amount of tissue liberated from the chamber, most or all of the islets are free of the surrounding acinar tissue, intact islets are observed, and the acinar tissue becomes finer (small cell clusters). Diphenylthiocarbazone (DTZ) staining can be used to distinguish islets from non-islet tissue. Scc, Latif et al., Transplantation 1988; 45:827-830, incorporated herein by reference. DTZ selectively binds to the zinc-insulin complex in islet beta cell granules, and results in a red staining of the islets. DTZ staining provides a rapid means for discrimination of islet from acinar tissue, and the positive reaction indicates that insulin-containing beta cells are present. During the collection phase, temperature of the system can be reduced to about 10° C. to about 30° C. Fluid in the fluid collecting flask can be allowed to flow through the pump and heat exchange coil into the Ricordi chamber, and Phase II solution (RPMI 1640, catalog #99-595-CM, Mediatech, Inc., Herndon, Va.) can be added to a loading flask. The Phase II solution can be pumped through the circulation system to dilute the collagenase and to wash the tissue. Digested material can be collected in flasks containing Phase II solution and human serum albumin (HSA), and the collected material washed two to five times using cold storage solution. Cold storage solution can include 16.00 to 20.00 g/L raffinose, 4.00 to 6.00 g/L histidine, 4.00 to 5.00 g/L sodium hydroxide, 30.00 to 40.00 g/L lactobionic acid, 0.30 to 0.50 g/L potassium hydroxide, 0.05 to 0.10 g/L calcium chloride, 1.00 to 1.50 g/L magnesium sulfate, 3.00 to 4.00 g/L sodium phosphate monobasic, 19.00 to 21.00 g/L pentastarch, 8.00 to 12.00 U/mL heparin, and 8.00 to 12.00 μg/mL insulin. For example, cold storage solution can include 17.84 g/L D (+) raffinose, 4.66 g/L L-histidine, 4.60 g/L sodium hydroxide, 35.83 g/L lactobionic acid, 0.39 g/L potassium hydroxide, 0.39 g/L calcium chloride dihydrate, 1.23 g/L magnesium sulfate heptahydrate, 3.45 g/L sodium phosphate monobasic, 2% penta starch, 10 U/mL heparin, and 10 μg/mL insulin. Cold storage solution can be made by combining H-Phase II solution (80% by volume) with 10% penta starch (i.e., 100 g/L) (20% by volume), and adding 8.00 to 12.00 U/mL heparin, and 8.00 to 12.00 μg/mL insulin. H-Phase II solution can include 16.00 to 20.00 g/L raffinose, 4.00 to 6.00 g/L histidine, 4.00 to 5.00 g/L sodium hydroxide, 30.00 to 40.00 g/L lactobionic acid, 0.30 to 0.50 g/L potassium hydroxide, 0.05 to 0.10 g/L calcium chloride, 1.00 to 1.50 g/L magnesium sulfate, and 3.00 to 4.00 g/L sodium phosphate monobasic. The pH of H-Phase II solution can be adjusted to a pH of 7.3-7.5 using hydrochloric acid or sodium hydroxide. Density of H-Phase II solution typically is 1.063±0.003. For example, H-Phase II solution can include 17.84 g/L D (+) raffinose, 4.66 g/L L-histidine, 4.60 g/L sodium hydroxide, 35.83 g/L lactobionic acid, 0.39 g/L potassium hydroxide, 0.39 g/L calcium chloride dihydrate, 1.23 g/L magnesium sulfate heptahydrate, and 3.45 g/L sodium phosphate monobasic. The washed tissue can be resuspended in capping layer solution and HSA (e.g., 25% HSA). Capping layer solution can include 16.00 to 20.00 g/L raffinose; 4.00 to 6.00 g/L histidine; 4.00 to 5.00 g/L sodium hydroxide; 30.00 to 40.00 g/L lactobionic acid; 0.30 to 0.50 g/L potassium hydroxide; 0.05 to 0.10 g/L calcium chloride; 1.00 to 1.50 g/L magnesium sulfate; 3.00 to 4.00 g/L sodium phosphate monobasic; and 19.00 to 21.00 g/L pentastarch. For example, capping layer solution can have a density of 1.035 to 1.036 g/cm³ and can include 17.84 g/L D (+) raffinose, 4.67 g/L L-Histidine, 4.6 g/L sodium hydroxide, 35.83 g/L lactobionic acid, 0.393 g/L potassium hydroxide, 0.07 g/L calcium chloride dihydrate, 1.23 g/L magnesium sulfate heptahydrate, 3.45 g/L sodium phosphate monobasic, and 2% penta starch. Capping layer solution can be made by combining H-Phase II solution (80% by volume) with 10% penta starch (i.e., 100 g/L) (20% by volume). Islets can be purified using continuous density gradient separation. Gradients can be prepared using iodixanol (OptiPrep™, Nycomed, Roskilde, Denmark) (density 1.32 g/cm³) and capping layer solution, cold storage solution, and/or high-density (HD) stock solution. HD stock solution can include 16.00 to 20.00 g/L raffinose; 4.00 to 6.00 g/L histidine; 4.00 to 5.00 g/L sodium hydroxide; 30.00 to 40.00 g/L lactobionic acid; 0.30 to 0.50 g/L potassium hydroxide; 0.05 to 0.10 g/L calcium chloride; 1.00 to 1.50 g/L magnesium sulfate; 3.00 to 4.00 g/L sodium phosphate monobasic; 15.00 to 25.00 g/L pentastarch; and 200 to 300 ml/L iodixanol. The density of the HD stock solution typically is 1.112±0.003 g/cm³. For example, HD stock solution can include 17.84 g/L D (+) raffinose, 4.67 g/L L-Histidine, 4.6 g/L sodium hydroxide, 35.83 g/L lactobionic acid, 0.39 g/L potassium hydroxide, 0.07 g/L calcium chloride dihydrate, 1.23 g/L magnesium sulfate heptahydrate, 3.45 g/L sodium phosphate monobasic, 20 g/L penta starch, and 250 mL/L iodixanol (Optiprep™). In some aspects, HD stock solution also can include 8.00 to 12.00 U/mL of heparin and/or 8.00 to 12.00 μg/mL insulin. A bottom density gradient solution having a density that ranges from 1.08 to 1.13 g/cm³ can be prepared by mixing HD stock solution and cold storage solution. A light density gradient solution having a density of 1.050 to 1.080 g/cm³ can be made by mixing iodixanol and cold storage solution, while a heavy density gradient solution having a density of 1.06 to 1.13 g/cm³ can be made by mixing cold storage solution and HD stock solution. A continuous gradient can be made, for example, in a dual chamber gradient maker, by combining the light and heavy density gradient solutions. The bottom density gradient can be transferred to a cell processing bag for a cell separator such as the Cobe 2991 cell separator (Lakewood, Colo.), and the continuous gradient can be overlaid on the bottom density gradient. The resuspended tissue (as described above) can be placed on the continuous gradient followed by a capping layer solution then the gradient can be spun to separate the islets. Fractions can be collected and assayed for the presence of islets as described below. Fractions with islet purities (percentage of DTZ positive cells)>10% can be combined for culture.

Purified islets can be cultured using a chemically defined culture medium that is effective for maintaining viability of human pancreatic islets under culture conditions. Typically, islets are cultured at a temperature of 22° C. to 37° C. and an atmosphere of 95% air and 5% CO₂. In some aspects, islets can be cultured in an atmosphere of room air. Viability of islets can be assessed using trypan blue or a fluorescent dye inclusion/exclusion assay. See, for example, Barnett et al., Cell Transplant. 2004; 13 (5): 481-8, incorporated herein by reference. The chemically defined culture medium can include one or more of the following: insulin, zinc sulfate, selenium, transferrin, sodium pyruvate, HEPES (N-[2-Hydroxyethyl]piperazine-N′ [2-ethanesulfonic acid]), HSA, and heparin. For example, the chemically defined culture medium can include 5.50 to 7.50 μg/mL insulin, 15 to 18 μM zinc sulfate, 5.50 to 7.50 ng/ml selenium (e.g., selenous acid), and 5.50 to 7.50 μg/mL transferrin (e.g., human transferrin). Such a culture medium further can include one or more of the following: 3 to 7 mM sodium pyruvate, 20 to 30 mM HEPES, 0.50 to 1.50 mg/mL HSA, 8.00 to 12.00 U/mL of heparin, 1 to 3 mM L-Alynyl-L-glutamine, and 4.50 to 6.50 μg/mL linoleic acid. Typically, when the cells are to be cultured under 95% room air and 5% CO₂, the chemically defined culture medium includes bicarbonate (e.g., 1.75 to 2.75 g/L such as 2.2 g/L). The bicarbonate concentration can be reduced if the cells are cultured in 100% room air. In some aspects, the chemically defined culture medium also includes an antibiotic such as ciprofloxacin (Bayer Corporation). In one aspect, a chemically defined culture medium can be CMRL 1066 (Mediatech, Inc., Herndon, Va.) supplemented with 25 mM HEPES, 2 mM L-Alynyl-L-Glutamine, 5 mM sodium pyruvate, 1% (vol/vol), ITS additive (6.25 μg/mL human recombinant insulin, 6.25 μg/mL human transferrin, 6.25 ng/mL selenous acid, 1.25 mg/mL HSA, 5.35 μg/mL linoleic acid), 16.7 μM zinc sulfate, 20 μg/mL ciprofloxacin (Bayer Corporation) and 0.5% final concentration of 25% HSA. Human Insulin-like Growth Factor-I (IGF-I, GRO PEP Pty Ltd, Adelaide, South Australia) can be added to the islet culture. For example, 90 to 110 ng/ml (e.g., 100 ng/ml) of IGF-1 can be added to the culture. Typically, the islets are cultured overnight at 37° C. then for an additional 1 to 3 days at 22° C. Pretransplant culture of islets can provide beneficial metabolic and immunologic effects. For example, culturing islets for two days can improve the metabolic efficacy of the cultured islets relative to freshly isolated islets. Pretransplant islet culture also can allow time for T-cell-directed immunosuppression to be achieved in the recipient before the transplant. Without being bound to a particular mechanism, achieving T-cell-directed immunosuppression may reduce islet-directed immune responses mediated by autoreactive, primed T cells to which the transplanted islets are immediately exposed. As described herein, delaying transplantation until two days after the initiation of therapy with T-cell-depleting antibodies prevents exposure of transplanted islets to the cytokine release associated, to varying degrees, with the first and second antibody infusions. Furthermore, pretransplant culture of islets allows quality control studies to be performed before the infusion of tissue.

Purified islet cells can be cryopreserved by suspending the cells in a cryopreservative such as dimethylsulfoxide (DMSO) or ethylene glycol, or a mixture of cryopreservatives. See, for example, Miyamoto et al., Cell Transplant 2001; 10 (4-5): 363-71; Evans et al., Transplantation 1990; 50 (2): 202-206; and Lakey et al., Cell Transplant 1996; 5 (3): 395-404, all of which are incorporated herein by reference. Islet cells can be cryopreserved after purification or culture. Typically, the cryopreservative is added in a stepwise fashion and the islets are slow cooled to −40° C. then stored at −196° C. Islets can be rapidly thawed (e.g., in a 37° C. water bath) and assayed before use. Cryopreservation can allow for long-term storage of these cells for later transplantation or other purposes. Cryopreserving collections of purified populations of islets cells is particularly useful for producing an islet bank.

Preparations of isogenic islet cells purified using the methods described herein typically result in successful transplants in at least 55% (e.g., at least 60%, 65%, 70%, 75%, 80%, 85%, 90%, or 95%) of the patients. A transplant is considered a success when a patient sustains insulin independence, normoglycemia, and freedom from hypoglycemia for at least 1, 2, 3, 4, 5, 6, 7, 8, 9, 10, 11, 12, 13, 14, 15, 16, 17, 18, 19, 20, or more weeks, or 1, 2, 3, 4, 5, 6, 7, 8, 9, 10 or more years after a single-donor islet transplant.

Preparations of purified islet cells can be assayed to confirm that the islets have sufficient potency to be used for the compositions and methods of the disclosure. As used herein, “transplant potency” refers to an estimate of the probability that the preparation of islets can be successfully transplanted in a patient and is based on one or more of the following parameters: safety of the islet preparation, islet cell number, cellular composition of islet preparation, number of beta cells, insulin content, tissue volume, viability, ATP content, percent of islet equivalents recovered after cell culture, percent necrotic and apoptotic cells, glucose-stimulated insulin release, and oxygen consumption rate (OCR). For example, transplant potency can be estimated based on the ATP/DNA ratio, OCR/DNA ratio, and beta cell number. Preparations of purified islets that have at least a 60% probability of constituting a successful transplant are particularly useful. Safety of an islet preparation can be determined by assaying for the presence of aerobic and anaerobic organisms and fungi, mycoplasma, and other adventitious agents (e.g., viruses) using known techniques. For example, a sample can be Gram stained to detect bacteria. Islet cells suitable for transplantation do not contain detectable organisms and are clinically sterile. Assessing safety also can include measuring endotoxin present in the preparation. Islet cell preparations suitable for transplant have an endotoxin content of 1.7 EU/mL (5 EU/kg recipient body weight) or less. Islet cell number can be assessed by staining with DTZ and quantifying the size distribution of the stained cells using a light microscope with ocular micrometer. Sec, Ricordi et al., Acta Diabetol. Lat. 1990; 27:185-195, incorporated herein by reference. Islet volume can be calculated, based on the assumption that islets are spherical, and the number of islets is expressed in terms of islet equivalents (IE), with one IE equal to a 150 μm diameter islet. Preparations of islets containing at least 2.2×10⁵ IE (e.g., 2.7×10⁵, 3.5×10⁵, 4.5×10⁵, 5.5×10⁵, 7.0×10⁵, 9.0×10⁵, 1.1×10⁶, or 1.4×10⁶ IE) are particularly useful as 5,000 to 20,000 IE can be transplanted/kg recipient body weight. One IE can include from about 600 to about 8,600 cells. The cellular composition of islet preparations can be assessed using standard immunoassay methods. Antibodies that have binding affinity for insulin, glucagon, somatostatin, pancreatic polypeptide, amylase, and cytokeratin 19 can be used to identify beta-, alpha-, delta-, pp-, acinar, and ductal cells, respectively. Such antibodies are commercially available, e.g., from DAKO, Carpinteria, Calif. or Sigma Chemical Co., St. Louis, Mo. Binding can be detected by labeling, either directly or indirectly, the antibody having binding affinity for the particular protein (e.g., insulin) or a secondary antibody that binds to such an antibody. Suitable labels include, without limitation, radionuclides (e.g., 125I, 131I, 35S, 3H, 32P, 33P, or 14C), fluorescent moieties (e.g., fluorescein, FITC, PerCP, rhodamine, or PE), luminescent moieties (e.g., Qdot™ nanoparticles supplied by the Quantum Dot Corporation, Palo Alto, Calif.), compounds that absorb light of a defined wavelength, or enzymes (e.g., alkaline phosphatase or horseradish peroxidase). Antibodies can be indirectly labeled by conjugation with biotin then detected with avidin or streptavidin labeled with a molecule described above. Methods of detecting or quantifying a label depend on the nature of the label and are known in the art. Examples of detectors include, without limitation, x-ray film, radioactivity counters, scintillation counters, spectrophotometers, colorimeters, fluorometers, luminometers, and densitometers. Immunological assays can be performed in a variety of known formats, including sandwich assays, competition assays (competitive RIA), or bridge immunoassays. See, for example, U.S. Pat. Nos. 5,296,347; 4,233,402; 4,098,876; and 4,034,074, all of which are incorporated herein by reference. The number of beta cells can be calculated based on the total DNA content and proportion of beta cells identified in the cellular composition sample. One IE can include from about 145 to 4,000 beta cells. Preparations of islet cells that contain at least 1×10⁶ beta cells/kg body weight of recipient (i.e., 4.5×10⁷ beta cells for a 45 kg recipient, 5×10⁷ beta cells for a 50 kg recipient, and 5.5×10⁷ beta cells for a 55 kg recipient) can be used. Preparations containing higher numbers of beta cells (e.g., at least 2×10⁶ beta cells/kg body weight of recipient, at least 3.5×10⁶ beta cells/kg body weight of recipient, or at least 5.0×10⁶ beta cells/kg body weight of recipient) are particularly useful. For example, preparations containing at least 3.5×10⁶ beta cells/kg body weight of recipient (i.e., about 1.58×10⁸ beta cells for a 45 kg recipient, about 1.75×10⁸ beta cells for a 50 kg recipient, and about 1.9×10⁸ beta cells for a 55 kg recipient) can sustain insulin independence for at least one year. Insulin content can be assessed using an immunoassay, e.g., the Human Insulin Enzyme Immunoassay (EIA) kit from Mercodia, Sweden, and corrected for the DNA content. Pico Green can be used to assess DNA content. In the Pico Green method, islet cells can be lysed with a solution containing ammonium hydroxide and a non-ionic detergent. Pico Green can be added to the sample and incubated in the dark. Samples are read on a fluorometer with an excitation of 480 nm and an emission of 520 nm and compared with a standard curve. Typically, one IE can include from about 4 to about 60 ng of DNA.

Tissue volume of the preparation refers to the volume of the islet cell pellet before transplant. Islet cells can be collected in a pre-weighed tissue culture flask and the islets can be allowed to sediment to a bottom corner of the flask over a period of time (e.g., 5 minutes). The medium can be removed from the flask and the mass recorded. Suitable preparations of islet cells have a volume of 10 mL or less (e.g., 8 mL or less, 7 mL or less, 5 mL or less, 3 mL or less, or 2 mL or less).

ATP content of islet cell preparations can be assessed via high performance liquid chromatography (HPLC) or by using an immunoassay (e.g., an ATP Determination Kit from Invitrogen Corp., Carlsbad, Calif.). In either method, samples can be prepared using the methods of Micheli et al. Clin. Chem. Acta 1993, 220:1-17 (incorporated herein by reference) in which trichloroacetic acid is used to extract the ATP and a freon/amine solution is used to neutralize the sample. Preparations of islet cells that have at least 76 pmol ATP/μg DNA (e.g., at least 80, 90, 100, 110, 150, 175, 190, or 193), as measured by HPLC, are particularly useful for transplants. A fluorescent dye inclusion/exclusion assay can be used to assess viability. Sec, for example, London et al., Hormone & Metabolic Research—Supplement 1990; 25:82-87, incorporated herein by reference. For example, fluorescein diacetate and propidium iodide (PI) can be used to assess viability. Fluorescein diacetate is dissociated by intracellular enzymes into free fluorescein, which fluoresces green under blue light excitation (490 nm) and provides evidence that the cells are alive and metabolically active. If the cell membrane has been damaged, PI can enter into the cell, intercalate into the nuclear DNA, and fluoresce red under green light excitation (545 nm). The proportion of green (viable) and red (dead) cells gives an indication of viability of the islet preparation. Alternatively, SYTO-13/ethidium bromide (SYTO/EB) and calcein AM/ethidium homodimer (C/EthD) fluorescent staining can be used to assess viability. Sec, for example, Barnett et al., Cell Transplant. 2004; 13 (5): 481-8, incorporated herein by reference. Preparations of islets that contain at least 70% (e.g., at least 75%, 80%, 85%, 90%, 95%, or 97%) viable cells are particularly useful for transplants.

The percent of IE recovered after culture can be determined using DTZ as described above. Preparations of islets in which at least 70% (e.g., least 75%, 80%, 85%, 90%, or 95%) of the IE were recovered after culture are particularly useful for transplants. The percent necrotic and apoptotic cells can be assessed using known methods. For example, apoptosis can be assessed by examining DNA fragmentation. For example, a Cell Death Detection ELISAPlus (Roche Biochemicals, Indianapolis, Ind.) can be used to detect cytoplasmic histone-associated DNA fragments. Preparations of islets in which 30% or less (e.g., 25%, 20%, 15%, 10%, 5%, or less) of the cells are apoptotic or necrotic are useful for transplants. Glucose-stimulated insulin release is a measure of the functional capacity of the preparation. Standard techniques for static incubation and assessment of insulin release corrected for DNA content are utilized to determine the functional capacity of the islets. See e.g., Ricordi et al., Acta Diabetol. Lat. 1990; 27:185-195, incorporated herein by reference. A stimulation index is calculated by dividing insulin release at 16.7 mM glucose by insulin release at 1.7 mM glucose. Preparations of islets that have a stimulation index of >1 (e.g., >4, >7, >10, >14, >17, or >27) are particularly useful for transplants. OCR can be measured using an OCR chamber (e.g., from Instech Laboratories, Inc., Plymouth Meeting, Pa.). See, for example, Papas et al., Cell Transplant. 2003; 12:177; Papas et al., Cell Transplant. 2003; 12:176; and Papas et al., Cell Transplant. 2001; 10:519, all of which are incorporated herein by reference. Preparations of islets having an OCR of greater than >75 nmol/min/mg DNA (e.g., greater than >100, >150, >200, or >230 nmol/min/mg DNA) are particularly useful for transplants.

### 2. Other Insulin Producing Cells

In some aspects, insulin-producing cells (e.g., beta cells) may be derived from another cell type, for example, through differentiation or trans-differentiation. In some aspects, insulin-producing cells (e.g., beta cells) are generated in vitro from a pluripotent cell, e.g., induced pluripotent stem cells, embryonic stem cells, non-embryonic stem cells, etc. In some aspects, a pluripotent cell is differentiated into an insulin-producing cell (e.g., beta cell) for use in methods of the disclosure.

### C. Cell Engineering

Certain aspects relate to cells comprising heterologous nucleic acids (e.g., vectors) of the disclosure. In some aspects, a cell is engineered to comprise expression or increased expression of a molecule of interest, such as a therapeutic agent. In some aspects, a cell is engineered to comprise expression or increased expression of extracellular matrix proteins. In some aspects, a cell is engineered to comprise expression or increased expression of a hormone (e.g., insulin). In some aspects, a cell is engineered to comprise expression or increased expression of an immunosuppressant agent (e.g., CD11b, CD1c, CD1a, CD4, CD14, CD19, CD24, CD25, CD27, CD33, CD38, CD15, CD66b, CD64, CD80, CD84, CD163, CD86, CD101, CD115, CD54, HLA-DR, CD177, CD317, PDL-1, CD170, LOX1, JAML, IRF7, RSAD2, IFIT1, B220, MHC-II, CD11c, Foxp3, FcεRI, CD206, CD172a). In some aspects, a cell is engineered to comprise reduced expression (e.g., knockdown or knockout) of a gene involved in immunosuppression (e.g., HLA-DR, CD15, CD14, CD170, CD10, CD16, CD127). Methods for producing cells comprising heterologous nucleic acids are well known in the art; see, for example, Sambrook et al. (2001, Molecular Cloning: A Laboratory Manual, Cold Spring Harbor Laboratory, New York), incorporated herein by reference.

### 1. Transduction

As used herein the term “transduce” or “transduction” refers to the process whereby a foreign nucleic acid sequence is introduced into a cell by means of a virus or viral vector. In some aspects, this transduction is done via a viral vector.

In certain aspects, the term “viral vector” intends a recombinant vector that retains the ability to infect and transduce nondividing and/or slowly-dividing cells and may integrate into the target cell's genome. In some aspects, the vector is derived from or based on a wild-type virus. In some aspects, the vector is derived from or based on a wild-type lentivirus. Examples of such include without limitation, human immunodeficiency virus (HIV), equine infectious anemia virus (EIAV), simian immunodeficiency virus (SIV) and feline immunodeficiency virus (Hy). Alternatively, it is contemplated that other retrovirus can be used as a basis for a vector backbone such murine leukemia virus (MLV). It will be evident that a viral vector according to the disclosure need not be confined to the components of a particular virus. The viral vector may comprise components derived from two or more different viruses, and may also comprise synthetic components. Viral vector components may be manipulated to obtain desired characteristics, such as target cell specificity.

The recombinant viral vectors of this disclosure may be derived from primates and non-primates. Examples of primate lentiviruses include the human immunodeficiency virus (HIV), the causative agent of human acquired immunodeficiency syndrome (AIDS), and the simian immunodeficiency virus (SIV). The non-primate lentiviral group includes the proto-type “slow virus” Visna-maedi virus (VMV), as well as the related caprine arthritis-encephalitis virus (CAEV), equine infectious anemia virus (EIAV) and the more recently described feline immunodeficiency virus (FIV) and bovine immunodeficiency virus (BIV). Recombinant lentiviral vectors are known in the art, e.g., see U.S. Pat. Nos. 6,924,123; 7,056,699; 7,419,829 and 7,442,551, all of which are incorporated herein by reference.

### 2. Transfection

As used herein, the term “transfection” is defined as the introduction of an extracellular nucleic acid into a host cell by any means known in the art, including calcium phosphate co-precipitation, viral transduction, liposome fusion, microinjection, microparticle bombardment, electroporation, etc. The terms “uptake of nucleic acid by a host cell”, “taking up of nucleic acid by a host cell”, “uptake of particles comprising nucleic acid by a host cell”, and “taking up of particles comprising nucleic acid by a host cell” denote any process wherein an extracellular nucleic acid, with or without accompanying material, enters a host cell.

A variety of methods are known in the art and suitable for transfection of nucleic acid into a cell. The nucleic acids of the present invention may be formulated, using the methods described herein. The formulations may contain nucleic acids which may be modified and/or unmodified. The formulations may further include, but are not limited to, cell penetration agents, a pharmaceutically acceptable carrier, a delivery agent, a bioerodible or biocompatible polymer, a solvent, and/or a sustained-release delivery depot.

The formulated nucleic acids may be delivered to the cell using routes of administration known in the art and described herein. Examples of typical methods include, but are not limited to, naked delivery, lipidoid-, liposome-, lipoplex-, and/or lipid nanoparticle-mediated transfer, electroporation, calcium phosphate mediated transfer, nucleofection, sonoporation, heat shock, magnetofection, microinjection, microprojectile mediated transfer (nanoparticles), cationic polymer mediated transfer (DEAE-dextran, polyethylenimine, polyethylene glycol (PEG) and the like) and/or cell fusion.

D. Extraction and/or Concentration of Cell-Derived Materials

Materials produced by the cells of the disclosure (e.g., CAFs and/or fibroblasts) may be extracted and/or concentrated using appropriate techniques, such as centrifugation, filtration, or chromatography. In this way, bioactive molecules produced by the cells of the disclosure, may be isolated and/or purified from the culture supernatant and/or cell lysate.

Certain aspects include production of one or more factors from CAF and/or fibroblast cultures through the use of filters that separate compositions based on electrical charge, size and/or ability to elute from an adsorbent. Numerous techniques are known in the art for purification of cell-derived factors and concentration of agents. For some particular uses CAF and/or fibroblast derived compounds are sufficient for use as culture supernatants of the cells in media. Currently media useful for this purpose include at least Roswell Park Memorial Institute (RPMI-1640), Dublecco's Modified Essential Media (DMEM), Eagle's Modified Essential Media (EMEM), Optimem, and Iscove's Media.

### 1. Concentration of Cell-Derived Materials

In certain aspect, CAF-derived and/or fibroblast-derived factors usable for methods or compositions of the disclosure may be derived from CAF and/or fibroblast cell cultures and may comprise exosomes, lysate, conditioned media, metabolites, vesicles, apoptotic bodies, any other secreted factor, or a combination thereof. In such an aspect, the factors may be concentrated by filtering/desalting means known in the art including use of ultracentrifugal membrane filters (e.g., Amicon® filters) with specific molecular weight cut-offs, said cut-offs may select for molecular weights higher than 1 kDa to 50 kDa. In some aspects, supernatant, or other compositions containing CAF-derived and/or fibroblast-derived factors, may be concentrated using means known in the art such as solid phase extraction using C18 cartridges (e.g., Mini-Spe-ed C18-14%, S.P.E. Limited, Concord ON). The cartridges may be prepared by washing with methanol followed by deionized-distilled water. For example, up to about 100 mL of CAF and/or fibroblast conditioned media supernatant may be passed through each of these specific cartridges before elution, and it is understood by one of skill in the art that larger or smaller cartridges may be used. After washing the cartridges, material adsorbed is eluted, such as with 3 mL methanol, evaporated under a stream of nitrogen, re-dissolved in a small volume of methanol, and optionally stored at 4° C. Before testing the eluate for activity in vitro and/or in vivo, the methanol is evaporated under nitrogen and replaced by culture medium. The C18 cartridges may be used to adsorb small hydrophobic molecules from the CAF conditioned supernatant and allows for the elimination of salts and other polar contaminants.

It may, however, be desired to use other adsorption means in order to purify certain factors. The concentrated supernatant, or other compositions containing CAF-derived factors and/or fibroblast-derived, may be assessed directly for biological activities useful for the practice of this disclosure, or may be further purified. Further purification may be performed using, for example, gel filtration using a Bio-Gel P-2 column with a nominal exclusion limit of 1800 Da (e.g., Bio-Rad, Richmond Calif.). Said column may be washed and pre-swelled in 20 mM Tris-HCl buffer, pH 7.2 (Sigma) and degassed by gentle swirling under vacuum. Bio-Gel P-2 material be packed into a 1.5×54 cm glass column and equilibrated with 3 column volumes of the same buffer. CAF and/or fibroblast cell supernatant concentrates, for example, may be extracted by C18 cartridge and dissolved in 0.5 ml of 20 mM Tris buffer, pH 7.2 and run through the column. Fractions may be collected from the column and analyzed for biological activity. Other purification, fractionation, and identification means are known to one skilled in the art and include anionic exchange chromatography, gas chromatography, high performance liquid chromatography, nuclear magnetic resonance, and mass spectrometry. Supernatant active fractions may be used in the compositions and/or methods disclosed herein.

### 2. Exosome Purification

Aspects of the disclosure include one or more compositions and methods of use thereof for the treatment of metabolic disease (e.g., diabetes), and in specific aspects the composition or methods of use may comprise exosomes from cultured CAFs and/or fibroblasts. In some aspects the exosomes are generated by a process comprising the steps of: a) obtaining one or more CAF and/or fibroblast cells; b) culturing said CAF and/or fibroblast cells under conditions to allow for production of exosomes into culture media within which said CAF and/or fibroblast cell is cultured; and c) obtaining exosomes from the culture media. In particular aspects the exosomes are administered to an individual in need of treatment. In particular aspects the exosomes are used to prepare one or more compositions of the disclosure. The CAFs and/or fibroblasts from which the exosomes are derived may be derived from a biopsy, wherein a donor providing the biopsy is either the individual to be treated (autologous) or the donor is different from the individual to be treated (allogeneic). In specific cases the CAFs and/or fibroblasts are cultured in a media allowing for CAF and/or fibroblast proliferation, and the media allowing for CAF and/or fibroblast proliferation may comprise one or more factors known to be mitogenic, such as one or more factors selected from the group consisting of: a) FGF-1; b) FGF-2; c) FGF-5; d) EGF; c) ciliary neurotrophic factor (CNTF); f) KGF-1; g) PDGF; h) platelet rich plasma; i) TGF-alpha; j) HGF-1; and k) a combination thereof.

Exosomes may or may not be obtained from CAFs and/or fibroblasts while the CAFs and/or fibroblasts are in a proliferating state. In some aspects, exosomes are obtained from CAFs and/or fibroblasts while the CAFs and/or fibroblasts are cultured in a media comprising no proliferative factors or largely reduced levels of proliferation inducing growth factors and the growth factors may be undefined growth factors such as fetal calf serum, neonatal serum, cord blood serum, or platelet lysate, or the growth factors may be defined mitogens such as EGF, FGF-1, FGF-2, FGF-5. In some aspects, exosomes may be obtained from CAFs and/or fibroblasts cultured under hypoxia. In specific aspects, exosomes are collected from CAFs and/or fibroblasts that have been cultured in 2-8% oxygen for at least 1 day. The amount of oxygen may be 2, 3, 4, 5, 6, 7, or 8% in the culture. A range of oxygen levels in culture may be 2-8, 2-7, 2-6, 2-5, 2-4, 2-3, 3-8, 3-7, 3-6, 3-5, 3-4, 4-8, 4-7, 4-6, 4-5, 5-8, 5-7, 5-6, 6-8, 6-7, or 7-8%. In specific aspects, the cells are cultured for 1-15 days or 5-10 days. The cells may be cultured for 1, 2, 3, 4, 5, 6, 7, 8, 9, 10, 11, 12, 13, 14, 15, 16, 17, 18, 19, or 20 or more days. The cells may be cultured for a range of days that is 1-15, 1-14, 1-13, 1-12, 1-11, 1-10, 1-9, 1-8, 1-7, 1-6, 1-5, 1-4, 1-3, 1-2, 2-15, 2-14, 2-13, 2-12, 2-11, 2-10, 2-9, 2-8, 2-7, 2-6, 2-5, 2-4, 2-3, 3-15, 3-14, 3-13, 3-12, 3-11, 3-10, 3-9, 3-8, 3-7, 3-6, 3-5, 3-4, 4-15, 4-14, 4-13, 4-12, 4-11, 4-10, 4-9, 4-8, 4-7, 4-6, 4-5, 5-15, 5-14, 5-13, 5-12, 5-11, 5-10, 5-9, 5-8, 5-7, 5-6, 6-15, 6-14, 6-13, 6-12, 6-11, 6-10, 6-9, 6-8, 6-7, 7-15, 7-14, 7-13, 7-12, 7-11, 7-10, 7-9, 7-8, 8-15, 8-14, 8-13, 8-12, 8-11, 8-10, 8-9, 9-15, 9-14, 9-13, 9-12, 9-11, 9-10, 10-15, 10-14, 10-13, 10-12, 10-11, 11-15, 11-14, 11-13, 11-12, 12-15, 12-14, 12-13, 13-15, 13-14, or 14-15 days. In culture, the cells may be passaged for at least 1, 2, 3, 4, 5, 6, 7, 8, 9, 10, or more passages. In specific cases the CAFs and/or fibroblasts are cultured in media selected from the group consisting of: a) Roswell Park Memorial Institute (RPMI-1640); b) Dublecco's Modified Essential Media (DMEM), c) Eagle's Modified Essential Media (EMEM), d) Optimem, and c) Iscove's Media.

In cases wherein exosomes from CAFs and/or fibroblasts are utilized, the exosomes may be in a preparation, such as a preparation that comprises less than 5% polyethylene glycol. The exosomes may be purified using polyethylene glycol and/or using ultrafiltration. In some cases, polyethylene glycol is added to the exosomes after purification. In some aspects, exosomes from CAFs may express one or more markers, such as CAFs comprise FAP, POSTN, PDGFRα/β, FSP-1, CD90, Palladin, OPN, AEBP1, Twist, TNC, Gallectin1, CD10 and GPR77, Cav-1, PDPN, CD200 In some aspects, the exosomes derived from fibroblasts may comprise expression of one or more of the following markers: CD63, CD9, major histocompatibility complex I (MHC I), or CD56.

In some aspects, CAFs and/or fibroblasts are cultured using means known in the art for preserving viability and proliferative ability of CAFs and/or fibroblasts. The disclosed methods may be applied both for individualized autologous exosome preparations and for exosome preparations obtained from established cell lines, for experimental or biological use. In some aspects, chromatography separation methods are used for preparing membrane vesicles, particularly to separate the membrane vesicles from potential biological contaminants, wherein the microvesicles are exosomes, and cells utilized for generating said exosomes are CAF and/or fibroblast cells. The exosomes are obtained and may be prepared for administration to one or more individuals in need thereof. The exosomes are obtained and may be prepared for use in one or more compositions of the disclosure. In some aspects CAF-derived and/or fibroblast-derived membrane vesicles, particularly exosomes, may be purified, and possess an ability to promote protection of a spheroid or organoid from an immune response. In some aspects, CAF-derived and/or fibroblast-derived membrane vesicles, e.g., exosomes, may be administered along with an organoid or spheroid of the disclosure. In some aspects, CAF-derived and/or fibroblast-derived membrane vesicles, e.g., exosomes, may be administered a site of previous administration of an organoid or spheroid of the disclosure. In some aspects, CAF-derived and/or fibroblast-derived membrane vesicles, e.g., exosomes, may be used to treat an organoid or spheroid of the disclosure ex vivo, e.g., during culture.

In some aspects, a strong or weak, preferably strong, anion exchange may be performed. In addition, in a specific aspect, the chromatography is performed under pressure. Thus, more specifically, it may consist of high performance liquid chromatography (HPLC). Different types of supports may be used to perform the anion exchange chromatography. In some embodiments, these may include cellulose, poly(styrene-divinylbenzene), agarose, dextran, acrylamide, silica, ethylene glycol-methacrylate co-polymer, or mixtures thereof, e.g., agarose-dextran mixtures. To illustrate this, it is possible to mention the different chromatography equipment composed of supports as mentioned above, particularly the following gels: SOURCE POROS® SEPHAROSE®, SEPHADEX®, TRISACRYL®, TSK-GEL SW OR PW®, SUPERDEX®TOYOPEARL HW and SEPHACRYL®, for example, which are suitable for the application of this invention. Therefore, in a specific aspect, this invention relates to a method of preparing membrane vesicles, particularly exosomes, from a biological sample such as a tissue culture containing CAFs, comprising at least one step during which the biological sample is treated by anion exchange chromatography on a support selected from cellulose, poly(styrene-divinylbenzene), silica, acrylamide, agarose, dextran, ethylene glycol-methacrylate co-polymer, alone or in mixtures, optionally functionalized.

In addition, to improve the chromatographic resolution, within the scope of the disclosure, one can use supports in bead form. In particular aspects, these beads have a homogeneous and calibrated diameter, with a sufficiently high porosity to enable the penetration of the objects under chromatography (i.e., the exosomes). In this way, given the diameter of exosomes (generally between 50 and 100 nm), to practice the invention, one can use high porosity gels, particularly between 10 nm and 5 μm, such as between approximately 20 nm and approximately 2 μm, including between about 100 nm and about 1 μm. For the anion exchange chromatography, the support used may be functionalized using a group capable of interacting with an anionic molecule. Generally, this group comprises an amine that may be ternary or quaternary, which defines a weak or strong anion exchanger, respectively. Within the scope of this disclosure, one can utilize a strong anion exchanger. In this way, according to the disclosure, a chromatography support as described above, functionalized with quaternary amines, may be used. Therefore, according to a more specific aspect of the disclosure, the anion exchange chromatography is performed on a support functionalized with a quaternary amine. In specific cases, this support is selected from poly(styrene-divinylbenzene), acrylamide, agarose, dextran and silica, alone or in mixtures, and may be functionalized with a quaternary amine. Examples of supports functionalized with a quaternary amine include the gels SOURCEQ. MONO Q, Q SEPHAROSE®, POROS®HQ and POROS® QE, FRACTOGEL®TMAE type gels and TOYOPEARL SUPER®Q gels.

One example of a support to perform the anion exchange chromatography comprises poly(styrene-divinylbenzene). An example of this type of gel that may be used within the scope of this disclosure is SOURCE Q gel, particularly SOURCE 15 Q (Pharmacia). This support offers the advantage of very large internal pores, thus offering low resistance to the circulation of liquid through the gel, while enabling rapid diffusion of the exosomes to the functional groups, which are particularly important parameters for exosomes given their size. The biological compounds retained on the column may be eluted in different ways, particularly using the passage of a saline solution gradient of increasing concentration, e.g. from 0 to 2 M. A sodium chloride solution may particularly be used, in concentrations varying from 0 to 2 M, for example. The different fractions purified in this way may be detected by measuring their optical density (OD) at the column outlet using a continuous spectro-photometric reading. As an indication, under the conditions used in the examples, the fractions comprising the membrane vesicles were eluted at an ionic strength comprised between approximately 350 and 700 mM, depending on the type of vesicles.

Different types of columns may be used to perform this chromatographic step, according to requirements and the volumes to be treated. For example, depending on the preparations, it is possible to use a column from approximately 100 μL up to 10 mL or greater. In this way, the supports available have a capacity which may reach 25 mg of proteins/mL, for example. For this reason, a 100 μL column has a capacity of approximately 2.5 mg of proteins which, given the samples in question, allows the treatment of culture supernatants of approximately 2 L (which, after concentration by a factor of 10 to 20, for example, represent volumes of 100 to 200 mL per preparation). It is understood that higher volumes may also be treated, by increasing the volume of the column, for example. In addition, to practice this invention, it is also possible to combine the anion exchange chromatography step with a gel permeation chromatography step. In this way, according to a specific aspect of the disclosure, a gel permeation chromatography step is added to the anion exchange step, either before or after the anion exchange chromatography step. In this aspect, the permeation chromatography step takes place after the anion exchange step. In addition, in a specific variant, the anion exchange chromatography step is replaced by the gel permeation chromatography step. The present application demonstrates that membrane vesicles may also be purified using gel permeation liquid chromatography, particularly when this step is combined with an anion exchange chromatography or other treatment steps of the biological sample, as described in detail below.

To perform the gel permeation chromatography step, a support selected from silica, acrylamide, agarose, dextran, ethylene glycol-methacrylate co-polymer or mixtures thereof, e.g., agarose-dextran mixtures, may be used. As an illustration, for gel permeation chromatography, a support such as SUPERDEX®200 HR (Pharmacia), TSK G6000 (TosoHaas) or SEPHACRYL®S (Pharmacia) may be used. The process according to the disclosure may be applied to different biological samples. In particular, these may comprise a biological fluid from a subject (bone marrow, peripheral blood, etc.), a culture supernatant, a cell lysate, a pre-purified solution or any other composition comprising membrane vesicles.

In this respect, in a specific aspect of the disclosure, the biological sample is a culture supernatant of membrane vesicle-producing CAF and/or fibroblast cells.

In addition, according to one aspect of the disclosure, the biological sample is treated, prior to the chromatography step, to be enriched with membrane vesicles (enrichment stage). In this way, in a specific aspect, this disclosure relates to a method of preparing membrane vesicles from a biological sample, characterized in that it comprises at least: a) an enrichment step, to prepare a sample enriched with membrane vesicles, and b) a step during which the sample is treated by anion exchange chromatography and/or gel permeation chromatography.

In one aspect, the biological sample is a culture supernatant treated so as to be enriched with membrane vesicles. In particular, the biological sample may be comprised of a pre-purified solution obtained from a culture supernatant of a population of membrane vesicle-producing cells or from a biological fluid, by treatments such as centrifugation, clarification, ultrafiltration, nanofiltration and/or affinity chromatography, particularly with clarification and/or ultrafiltration and/or affinity chromatography. Therefore, one method of preparing membrane vesicles according to this disclosure more particularly comprises the following steps: a) culturing a population of membrane vesicle (e.g., exosome) producing cells under conditions enabling the release of vesicles, b) a step of enrichment of the sample in membrane vesicles, and c) an anion exchange chromatography and/or gel permeation chromatography treatment of the sample.

As indicated above, the sample (e.g., supernatant) enrichment step may comprise one or more centrifugation, clarification, ultrafiltration, nanofiltration and/or affinity chromatography steps on the supernatant. In some aspects, the enrichment step comprises (i) the elimination of cells and/or cell debris (clarification), optionally followed by (ii) a concentration and/or affinity chromatography step. In one specific aspect, the enrichment step comprises an affinity chromatography step, optionally preceded by a step of elimination of cells and/or cell debris (clarification). An example of an enrichment step according to this disclosure comprises (i) the elimination of cells and/or cell debris (clarification), (ii) a concentration and (iii) an affinity chromatography. The cells and/or cell debris may be eliminated by centrifugation of the sample, for example, at a low speed, such as below 1000 g, between 100 and 700 g, for example. Certain centrifugation conditions during this step are approximately 300 g or 600 g for a period between 1 and 15 minutes, for example.

The cells and/or cell debris may also be eliminated by filtration of the sample, possibly combined with the centrifugation described above. The filtration may particularly be performed with successive filtrations using filters with a decreasing porosity. For this purpose, filters with a porosity above 0.2 μm, e.g., between 0.2 and 10 μm, may be used. It is particularly possible to use a succession of filters with a porosity of 10 μm, 1 μm, 0.5 μm followed by 0.22 μm.

A concentration step may also be performed, such as in order to reduce the volumes of sample to be treated during the chromatography stages. In this way, the concentration may be obtained by centrifugation of the sample at high speeds, e.g., between 10,000 and 100,000×g, to cause the sedimentation of the membrane vesicles. This may comprise a series of differential centrifugations, with the last centrifugation performed at approximately 70,000×g. The membrane vesicles in the pellet obtained may be taken up with a smaller volume and in a suitable buffer for the subsequent steps of the process. The concentration step may also be performed by ultrafiltration. In fact, this ultrafiltration allows both to concentrate the supernatant and perform an initial purification of the vesicles. According to a particular aspect, the biological sample (e.g., the supernatant) is subjected to an ultrafiltration, such as a tangential ultrafiltration. Tangential ultrafiltration comprises concentrating and fractionating a solution between two compartments (filtrate and retentate), separated by membranes of determined cut-off thresholds. The separation is carried out by applying a flow in the retentate compartment and a transmembrane pressure between this compartment and the filtrate compartment. Different systems may be used to perform the ultrafiltration, such as spiral membranes (Millipore, Amicon), flat membranes or hollow fibers (Amicon, Millipore, Sartorius, Pall, GF, Sepracor). Within the scope of the disclosure, the use of membranes with a cut-off threshold below 1000 kDa, such as between 300 kDa and 1000 kDa, or such as between 300 kDa and 500 kDa, is advantageous.

The affinity chromatography step can be performed in various ways, using different chromatographic support and material. It is advantageously a non-specific affinity chromatography, aimed at retaining (i.e., binding) certain contaminants present within the solution, without retaining the objects of interest (i.e., the exosomes). It is therefore a negative selection. In some cases, an affinity chromatography on a dye may be used, allowing the elimination (i.e., the retention) of contaminants such as proteins and enzymes, for instance albumin, kinases, dehydrogenases, clotting factors, interferons, lipoproteins, or also co-factors, etc. In certain embodiments, the support used for this chromatography step is a support as used for the ion exchange chromatography, functionalized with a dye. As a specific example, the dye may be selected from Blue SEPHAROSE® (Pharmacia), YELLOW 86, GREEN 5 and BROWN 10 (Sigma). The support may be agarose. It should be understood that any other support and/or dye or reactive group allowing the retention (binding) of contaminants from the treated biological sample can be used in the instant disclosure.

In one aspect a membrane vesicle preparation process within the scope of this disclosure comprises the following steps: a) the culture of a population of membrane vesicle (e.g., exosome) producing cells under conditions enabling the release of vesicles, b) the treatment of the culture supernatant with at least one ultrafiltration or affinity chromatography step, to produce a biological sample enriched with membrane vesicles (e.g., with exosomes), and c) an anion exchange chromatography and/or gel permeation chromatography treatment of the biological sample. In a certain aspect, step b) above comprises a filtration of the culture supernatant, followed by an ultrafiltration, such as tangential. In another aspect, step b) above comprises a clarification of the culture supernatant, followed by an affinity chromatography on dye, such as on Blue SEPHAROSE®. In addition, after step c), the material harvested may, if applicable, be subjected to one or more additional treatment and/or filtration stages d), particularly for sterilization purposes. For this filtration treatment stage, filters with a diameter less than or equal to 0.3 μm may be used, or for example, less than or equal to 0.25 μm. Such filters have a diameter of 0.22 μm, for example. After step d), the material obtained is, for example, distributed into suitable devices such as bottles, tubes, bags, syringes, etc., in a suitable storage medium. The purified vesicles obtained in this way may be stored cold, frozen or used extemporaneously. Therefore, a specific preparation process within the scope of the disclosure comprises at least the following steps: c) an anion exchange chromatography and/or gel permeation chromatography treatment of the biological sample, and d) a filtration step, particularly sterilizing filtration, of the material harvested after stage c). In a first variant, the process according to the disclosure comprises: c) an anion exchange chromatography treatment of the biological sample, and d) a filtration step, particularly sterilizing filtration, on the material harvested after step c). In some cases, instead of being stored the material may be used for one or more individuals in the absence of a prior storage step. In another variant, the process according to the disclosure comprises: c) a gel permeation chromatography treatment of the biological sample, and d) a filtration step, particularly sterilizing filtration, on the material harvested after step c). According to a third variant, the process according to the disclosure comprises: c) an anionic exchange treatment of the biological sample followed or preceded by gel permeation chromatography, and d) a filtration step, particularly sterilizing filtration, on the material harvested after step c).

### E. Pharmaceutical Compositions

In some aspects, pharmaceutical compositions of the present disclosure comprise an effective amount of one or more compositions comprising CAFs, fibroblasts, FECM, CECM, secretory cells (e.g., insulin producing cells), products derived from CAFs, product derived from fibroblasts, or a combination thereof dissolved or dispersed in a pharmaceutically acceptable carrier. The phrases “pharmaceutical” and “pharmacologically acceptable” and used interchangeably herein refer to molecular entities and compositions that do not produce an adverse, allergic or other untoward reaction when administered to a subject, such as, for example, a human, as appropriate, and do not interfere with the therapeutic methods of the disclosure. The preparation of a pharmaceutical composition that comprises CAFs, fibroblasts, FECM, CECM, secretory cells (e.g., insulin producing cells), products derived from CAFs, products derived from fibroblasts, or a combination thereof, or additional active ingredient(s), will be known to those of skill in the art in light of the present disclosure, as exemplified by Remington: The Science and Practice of Pharmacy, 21st Ed. Lippincott Williams and Wilkins, 2005, specifically incorporated by reference herein in its entirety. Moreover, for administration to an individual, it will be understood that preparations should meet sterility, pyrogenicity, general safety, and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated by reference herein in its entirety). Except insofar as any conventional carrier is incompatible with the active ingredient, its use in the pharmaceutical compositions is contemplated. The compositions comprising CAFs, fibroblasts, FECM, CECM, secretory cells (e.g., insulin producing cells), products derived from CAFs, products derived from fibroblasts, or a combination thereof may comprise different types of carriers depending on the form in which the composition is to be administered.

In certain aspects, the compositions and/or agents in some aspects of the disclosure may be formulated into preparations for local delivery (i.e., to a specific location of the body) or systemic delivery, in solid, semi-solid, gel, liquid or gaseous forms such as tablets, capsules, powders, granules, ointments, solutions, depositories, and injections allowing for surgical administration or implantation.

The carrier may be assimilable and include liquid, semi-solid, i.e., pastes, or solid carriers. Examples of carriers or diluents include fats, oils, water, saline solutions, lipids, liposomes, resins, binders, fillers, alcohols, and the like, or combinations thereof. Suitable carriers include, for example, distilled water, physiological phosphate-buffered saline, normal or lactated Ringer's solutions, dextrose solution, Hank's solution, or propanediol. The carrier and agent may be compounded as a liquid, suspension, polymerizable or non-polymerizable gel, paste or salve. The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles. In addition, sterile, fixed oils may be employed as a solvent or suspending medium. For this purpose, any biocompatible oil may be employed including synthetic mono- or di-glycerides. In addition, fatty acids such as oleic acid find use in the preparation of injectables.

A pharmaceutical composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, a composition of the disclosure is combined with a carrier or solvent in any convenient and practical manner, i.e., by solution, suspension, emulsification, admixture, enveloping, absorption and the like. Such procedures are routine for those skilled in the art.

In a specific aspect of the present disclosure, the composition is combined or mixed thoroughly with a semi-solid or solid carrier. Stabilizing agents can be also added in the mixing process in order to protect the composition from loss of biological activity. Examples of stabilizers for use in the composition include buffers, amino acids such as glycine and lysine, carbohydrates such as dextrose, mannose, galactose, fructose, lactose, sucrose, maltose, sorbitol, mannitol, etc.

In further aspects, the present disclosure may include the use of a pharmaceutical lipid vehicle compositions that incorporate compositions comprising CAFs, fibroblasts, DECM, CECM, secretory cells (e.g., insulin producing cells), products derived from CAFs, products derived from fibroblasts, or a combination thereof, one or more lipids, and an aqueous solvent. As used herein, the term “lipid” will be defined to include any of a broad range of substances that is characteristically insoluble in water and extractable with an organic solvent. This broad class of compounds is well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present disclosure.

One of ordinary skill in the art would be familiar with the range of techniques that can be employed for dispersing a composition in a lipid vehicle. For example, the composition(s) of the disclosure may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The composition(s) of the disclosure may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as, for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective.

In certain aspects, the pharmaceutical compositions are administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

### 1. Administration

In some aspects, administration of a pharmaceutical composition of the disclosure may be accomplished via any common route so long as the target tissue is available via that route. This may include intravenously, intramuscularly, subcutaneously, topically, orally, transdermally, intraperitoneally, intraorbitally, by implantation, by inhalation, by injectable insertion, intrathecally, intraventricularly, and/or intranasally. In some aspects, administration of a pharmaceutical composition of the disclosure may be accomplished via injectable insertion or implantation. Such compositions would normally be administered as pharmaceutically acceptable compositions that include physiologically acceptable carriers, buffers or other excipients as described elsewhere in this disclosure.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

In some aspects, a single administration of a composition (e.g., organoid) is provided. In some aspects, multiple administrations are provided. In some aspects, multiple administrations are provided over the course of 3-7 consecutive days. In some aspects, 3-7 administrations are provided over the course of 3-7 consecutive days. In some aspects, 5 administrations are provided over the course of 5 consecutive days. In some aspects, a single administration of between about 10⁵ and about 10¹³ cells per 100 kg is provided. In some aspects, a single administration of between about 1.5×10⁸ and about 1.5×10¹² cells per 100 kg is provided. In some aspects, a single administration of between about 1×10⁹ and about 5×10¹¹ cells per 100 kg is provided. In some aspects, a single administration of about 5×10¹⁰ cells per 100 kg is provided. In some aspects, a single administration of 1×10¹⁰ cells per 100 kg is provided. In some aspects, multiple administrations of between about 10⁵ and about 10¹³ cells per 100 kg are provided. In some aspects, multiple administrations of between about 1.5×10⁸ and about 1.5×10¹² cells per 100 kg are provided. In some aspects, multiple administrations of between about 1×10⁹ and about 5×10¹¹ cells per 100 kg are provided over the course of 3-7 consecutive days. In some aspects, multiple administrations of about 4×10⁹ cells per 100 kg are provided over the course of 3-7 consecutive days. In some aspects, multiple administrations of about 2×10¹¹ cells per 100 kg are provided over the course of 3-7 consecutive days. In some aspects, 5 administrations of about 3.5×10⁹ cells are provided over the course of 5 consecutive days. In some aspects, 5 administrations of about 4×10⁹ cells are provided over the course of 5 consecutive days. In some aspects, 5 administrations of about 1.3×10¹¹ cells are provided over the course of 5 consecutive days. In some aspects, 5 administrations of about 2×10¹¹ cells are provided over the course of 5 consecutive days.

### F. Generation of Compositions and Methods of Use

A composition comprising encapsulated cells (e.g., CAF and/or fibroblast encapsulated cells) may be generated by any suitable method. In certain aspects, tencapsulation is achieved by an in vitro method of creating spheroids from any single type of cell (e.g., CAFs and/or fibroblasts), a combination of two or more type of cell types (e.g., CAFs and insulin producing cells), secreted ECM (e.g., CEMC and/or DECM), or a combination thereof. Spheroids can be produced by culturing cells in pipette tips, microwells, or microcavities. These methods are adaptable to scale up production of cell spheroids for scientific research and translational applications.

In some aspects, spheroids comprise a cell capable of inducing immunosuppression. In some aspects, a cell with immunosuppressive effects may comprise one or more of the following expression profiles: CD11b⁺CD14⁺CD33⁺HLA-DR⁻CD15⁻; CD11b⁺CD14⁺CD33⁺HLA-DR⁻CD15⁺; CD11b⁺CD14⁻CD15⁺CD33⁺HLA-DR⁻; CD11b⁺CD14⁻CD66b⁺; CD11b⁺CD14⁻CD15⁻CD33⁺HLA-DR⁻; CD64⁺CD80⁺; CD163⁺CD86⁺; CD11b⁺CD66b⁺CD101⁺CD54⁺HLADR⁺CD86⁺CD15^{high}CD170^{low}; CD177⁺; CD11b⁺CD66b⁺PDL1⁺CD170^{high}; CD11b⁺CD66b⁺CD117⁺CD10⁻CD16^{int/low}LOX1⁺CD84⁺JAML⁺; CD11b⁺CD66b⁺; IFIT1⁺; IRF7⁺; RSAD2⁺; B220⁺CD11c^{low}MHC-II⁺CD317⁺; CD11c⁺CD115⁺CD1c⁺CD1a⁺FcεRI⁺CD206⁺CD172a⁺CD14⁺CD11b⁺; CD4⁺CD25⁺Foxp3⁺CD127^{lo/-}; CD4⁺CD25⁺Foxp3⁺CD127^{lo/-}; CD19⁺CD24^{hi}CD27⁺; CD19⁺CD24⁺CD38⁺. Cells with immunosuppressive capabilities are described in Tie Y, Tang F, Wei Y Q, Wei X W. Immunosuppressive cells in cancer: mechanisms and potential therapeutic targets. J Hematol Oncol. 2022 May 18; 15 (1): 6, which is incorporated in its entirety by reference herein.

Aspects of methods for creating cell spheroids with complex multicellular structures (i.e., core-shell structure) inside pipette tips and efficiently ensuring their survival rate and biological activity are encompassed herein. In specific aspects, it comprises the following three steps:
1. Making the core spheroid with any type of cell (e.g., pancreatic beta cells) in a single pipette tip and culturing for a suitable duration of time, (e.g., about 48 hours);
2. Generating a second layer (i.e., shell) on the spheroids with CAF cells in the same pipette tip and culture for about 48 hours. The process can be repeated to add additional layers of cell types to the spheroid.
3. Finally, after the cells spheroids form in the pipette tip for about 96 hours, the cell spheroids are collected from the pipette tip by ejecting the liquid containing the spheroid from inside the pipette tip, and removing the liquid matrix using centrifugation or filtration, and collecting the compositions.

Aspects of methods for creating cell spheroids with complex multicellular structures (i.e., core-shell structure) inside microwells or microcavities and efficiently ensuring their survival rate and biological activity are encompassed herein. In specific aspects, it comprises the following steps:
1) Generating a suitable amount of cells (e.g., CAFs, fibroblasts, and/or beta cells) in a two-dimensional environment (e.g., flat cell culture plates).
2) Culturing a suitable amount of cells of any type of cell (e.g., CAFs, fibroblasts, and/or beta cells) in a microwell or microcavity for about 72 hours to generate a spheroid.
3) Collecting the spheroid by washing with a physiological solution (e.g., PBS), removing the liquid matric using centrifugation and/or filtration, and collecting the spheroids.

Aspects of the disclosure relate to methods of treating a disease or disorder using one or more compositions of the disclosure. In some aspects, a disease or disorder to be treated may be a metabolic disorder (e.g., diabetes). In some aspects, one or more compositions of the disclosure are used for administration to an individual diagnosed with, suspected of having, or showing symptoms of having a disease or disorder (e.g., diabetes) to cure, treat, or ameliorate the disease or disorder. In some aspects, pancreatic beta cells encapsulated by CAFs, fibroblasts, FECM, and/or CECM are administered to an individual to treat diabetes.

Encapsulated cells (e.g., insulin producing cells) can be administered to an individual, for example, through the portal vein of the individual using surgical techniques such as minilaparotomy or percutaneous transhepatic portal venous catheterization. Prior to administration, patients may undergo induction of immunosuppression using different therapy regimens. Patients also may also undergo post-transplant immunosuppression regimens. For example, induction therapy can include treatment with rabbit antithymocyte globulin (RATG), daclizumab, and etanercept (i.e., soluble tumor necrosis factor (TNF) receptor). RATG is a potent induction agent and also interferes with leukocyte responses to chemotactic signals and inhibits the expression of integrins required for firm cellular adhesion. Selective inhibition of TNFα in the peritransplant period may be able to promote reversal of diabetes after marginal-mass islet transplants. Post-administration, the function of engrafted encapsulated cells (e.g., insulin producing cells) may be enhanced by replacing or minimizing tacrolimus at 1 month post-transplant. Another example of an induction therapy can include use of anti-CD3 mAb hOKT3γ1 (Ala-Ala), which can inactivate autoreactive, primed, islet-directed T cells immediately posttransplant. Anti-CD3 mAb, hOKT3γ1 (Ala-Ala), is a humanized antibody that retains the binding region of OKT3 but replaces the murine framework with human amino acids. In addition, the human IgG1 Fc is mutated to prevent binding to the Fc receptor (FcR). Clinically, this engineered antibody has proven effective in preserving residual beta-cell function in new-onset type 1 diabetes. In addition, the hOKT3γ1 (Ala-Ala) reversed kidney graft rejection. This dual activity against both autoreactive and alloreactive T cell responses occurred with markedly fewer side effects, as compared with the parental OKT3 antibody. In some aspects, the patient does not require immunosuppression.

In aspects of the disclosure, CAFs, fibroblasts, FECM, CECM, or a combination thereof, are used to enhance tolerance of allogeneic, autologous, syngeneic, and/or xenogeneic cells (e.g., insulin producing cells). CAFs and/or fibroblasts may be used in an unmanipulated manner, or manipulated by culture conditions, or may be genetically manipulated. Genetic manipulation may involve augmentation of immune suppressive/immune modulatory aspects and/or enhanced expression or production of ECM components.

In some aspects of the invention transfection is accomplished by use of lentiviral vectors, said means to perform lentiviral mediated transfection are well-known in the art. Some specific examples of lentiviral based transfection of genes into adherent cells include transfection of SDF-1 to promote stem cell homing, or growth factors such as FGF-18, HGF, akt, TRAIL, PGE-1, NUR77 to enhance migration, BDNF, HIF-1 alpha, CCL2, interferon beta, HLA-G to enhance immune suppressive activity, hTERT, cytosine deaminase, OCT-4 to reduce senescence, BAMBI to reduce TGF expression, HO-1 for antiapoptosis, LIGHT, miR-126 to enhance angiogenesis, bcl-2 to prevent apoptosis, telomerase and myocardin to induce cardiogenesis, CXCR4 to accelerate hematopoietic recovery and reduce renal allograft rejection, wnt11, Islet-1 to promote pancreatic differentiation, IL-27 to reduce autoimmune disease, ACE-2 to reduce sepsis, CXCR4 to reduce liver failure, and/or the HGF antagonist NK4 to reduce cancer.

In some aspects, the quality of CAF and/or fibroblast cultures may be determined before use for methods of the disclosure, for example flow cytometry may be performed on cultures to assess expression of SH-2, SH-3, SH-4 MSC markers and lack of contaminating CD14- and CD-45 positive cells. In some aspects, cells are detached with 0.05% trypsin-EDTA, washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG (H+L) antibody. In some aspects, cell cultures may be tested for sterility weekly, endotoxin by limulus amebocyte lysate test, and mycoplasma by DNA-fluorochrome stain.

Cultured CAFs and/or fibroblasts may be expanded and stored for later use. For example, confluent CAFs and/or fibroblasts in 175 cm² flasks are washed with Tyrode's salt solution, incubated with medium 199 (M199) for 60 min, and detached with 0.05% trypsin-EDTA (Gibco). Cells from 10 flasks were detached at a time and CAFs and/or fibroblasts are resuspended in 40 ml of M199+1% human serum albumin (HSA; American Red Cross, Washington DC, USA). CAFs and/or fibroblasts harvested from each 10-flask set were stored for up to 4 h at 4° C. and combined at the end of the harvest. A total of 2-10×10⁶ cells/kg are resuspended in M199+1% HSA and centrifuged at 460×g for 10 min at 20° C. Cell pellets are resuspended in fresh M199+1% HSA media and centrifuged at 460×g for 10 min at 20° C. for three additional times. Total harvest time is 2-4 h based on CAF yield per flask and the target dose. Harvested CAFs and/or fibroblasts may be cryopreserved, e.g., in Cryocyte (Baxter, Deerfield, IL, USA) freezing bags, using a rate controlled freezer with a final concentration of 10% DMSO (Research Industries, Salt Lake City, UT, USA) and 5% HSA.

Prior to encapsulation, cryopreserved CAF and/or fibroblasts units are thawed at 37° C. in a water bath and transferred to pre-formed organoid cores. CAFs and/or fibroblasts may be thawed 10, 9, 8, 7, 6, 5, 4, 3, 2, 1, or 0 days prior to encapsulation. In certain cases, the CAFs and/or fibroblasts are treated ex vivo to secrete ECM by treating with one or more, or combination of more than one of hypoxic conditions, oxidative stress, and certain growth factors produced by tumor cells, e.g., TGF-β, epidermal growth factor (EGF), fibroblast growth factor type 2 (FGF2), PDGF, Activin A, Nodal, BRAF inhibitors such as Vemurafenib, dabrafenib, and encorafenib. In some aspects, the CFAs and/or fibroblasts are activated with cytokines, chemokines, growth factors, transcription factors, nucleic acids, to secrete ECM. The CFAs and/or fibroblasts may be activated with CAF and/or fibroblasts exosomes, CAF and/or fibroblasts lysates, CAF and/or fibroblasts extracellular vesicles or other clinically beneficial CAFs and/or fibroblast fragments to secrete ECM. In some aspects, the CAFs and/or fibroblasts are engineered using CRISPR to secrete ECM, and they may be engineered using CRISPR to secrete ECM when activated in vivo. In some aspects, the CAFs and/or fibroblasts are used to encapsulate any cells or combination or one or more cell types producing and secreting one or more desired proteins, enzymes, or metabolites. In some aspects, the CAFs, fibroblasts, FECM and/or CECM are used to encapsulate any cells producing and secreting a desired protein, enzyme, or metabolite. For example, the CAFs and/or fibroblasts, whether pre-activated or not, may be added to a pre-formed core of cells (e.g., insulin producing cells) and allowed to form an outer layer around the core of cells, which may require incubation for 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 minutes or more, or any range derivable therein; for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 days or more, or any range derivable therein.

On the day of implantation, core-shell organoids are released from their encapsulation device (e.g., a pipette tip) and combined with a pharmaceutical acceptable carrier. The pharmaceutical composition containing the core-shell organoids is administered to the individual as appropriate (e.g., via injectable insertion). Individuals may be medicated pre- and post-implantation as required by a physician and/or surgeon. Blood pressure, pulse, respiratory rate, temperature and oxygen saturation are monitored at the time of implantation and every 15 min thereafter for 3 h followed by every 2 h for 6 h.

In some aspects, FECM and/or CECM may be used for encapsulation of any cells producing and secreting a desired protein, enzyme, or metabolite. In some aspects, FECM and/or CECM may be used as an attachment substrate for any cells producing and secreting a desired protein, enzyme, or metabolite.

CAFs and/or fibroblasts may be autologous, allogeneic, syngeneic, or xenogeneic with respect to an individual. In some aspects, the cells being encapsulated by CAFs and/or fibroblasts are autologous, allogeneic, syngeneic, or xenogeneic with respect to an individual. In some aspects, the CAFs, fibroblasts, FECM, CEMC, CAF- and/or fibroblasts-encapsulated cells, CAF and/or fibroblasts exosomes, CAF and/or fibroblasts lysates, CAF and/or fibroblasts extracellular vesicles or other clinically beneficial CAF or fibroblast fragments may be autologous, allogeneic, syngeneic, or xenogeneic with respect to an individual.

Certain aspects include a kit comprising CAF- and/or fibroblasts-encapsulated pancreatic beta cells, CAF and/or fibroblasts exosomes, CAF 1 and/or fibroblasts lysates, CAF and/or fibroblasts extracellular vesicles or other clinically beneficial CAFs or fibroblast fragments along with instrumentation required to perform a method of the disclosure.

Although the present disclosure and its advantages have been described in detail, it should be understood that various changes, substitutions and alterations can be made herein without departing from the spirit and scope of the invention as defined by the appended claims. Moreover, the scope of the present application is not intended to be limited to the particular aspects of the process, machine, manufacture, composition of matter, means, methods and steps described in the specification. As one of ordinary skill in the art will readily appreciate from the disclosure of the present disclosure, processes, machines, manufacture, compositions of matter, means, methods, or steps, presently existing or later to be developed that perform substantially the same function or achieve substantially the same result as the corresponding aspects described herein may be utilized according to the present disclosure. Accordingly, the appended claims are intended to include within their scope such processes, machines, manufacture, compositions of matter, means, methods, or steps.

### EXAMPLES

The following examples are included to demonstrate particular aspects of the invention. It should be appreciated by those of skill in the art that the techniques disclosed in the examples which follow represent techniques discovered by the inventor to function well in the practice of the invention, and thus can be considered to constitute particular modes for its practice. However, those of skill in the art should, in light of the present disclosure, appreciate that many changes can be made in the specific aspects which are disclosed and still obtain a like or similar result without departing from the spirit and scope of the invention.

### Example 1—Methods

Formation of mixed spheroids with CAF, HDF, or pancreatic fibroblasts with pancreatic beta cells was performed as follows:
1) Each cell type is grown to yield sufficient numbers (e.g., 500-10,000 cells or more) in a two-dimensional condition (e.g., in flat-surface culture plates).
2) Once cultures have reached a desired confluency, the cells are detached from the plates (e.g., by trypsinization), washed in buffer saline, and counted.
3) The cells to be encapsulated (e.g., pancreatic beta cells) are mixed with the encapsulating cells (e.g., CAF, HFD, or pancreatic fibroblasts) at various ratios (e.g., pancreatic beta cells-to-encapsulating cells at a ratio ranging from 1:100 to 100:1) in culture media.
4) Various amounts of the cell mixture (e.g., 500 to 10,000 cells) are plated in a cell culture plate suitable for promoting spheroid formation (e.g., a well of an ultra-low attachment 96-well plates, a plate with a microcavity, or a plate with a microwell).
5) The plate is spun in a centrifuge at 200×g for 3 minutes.
6) The plate is incubated at 37° C. in a CO₂ incubator for a time sufficient for the spheroids to form (e.g., about 72 hours).
7) Once the spheroids have formed, they are collected for downstream applications.

### Example 2—Generation of Cell Spheroids

Spheroids were prepared as described in Example 1. Plated cell mixtures were imaged immediately after the centrifugation step (Day 0) and again after 3 days of incubation at 37° C. in a CO₂ incubator (Day 3). The following groups were tested: fibroblasts only, beta cells only, and mixtures of beta cells to fibroblasts at 1:4, 1:1, and 4:1 ratios.

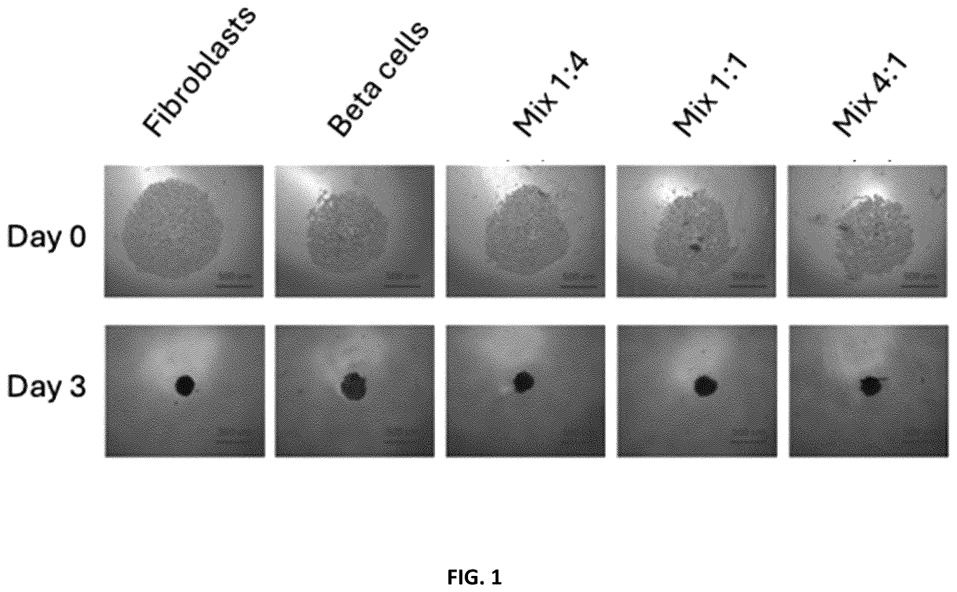

The results showed that each group formed clear compactization of cells into spheroids at Day 3 (FIG. 1).

### Example 3—Generation of Insulin-Producing Cell Spheroids

Spheroids comprising fibroblasts alone, beta cells alone, and a mixture of beta cells to fibroblasts at a 1:1 ratio were prepared as described in Example 1. Spheroids were stained for immunofluorescence imaging of nuclei (DAPI), insulin (beta cell marker), and vimentin (fibroblast marker) and imaged at Day 3.

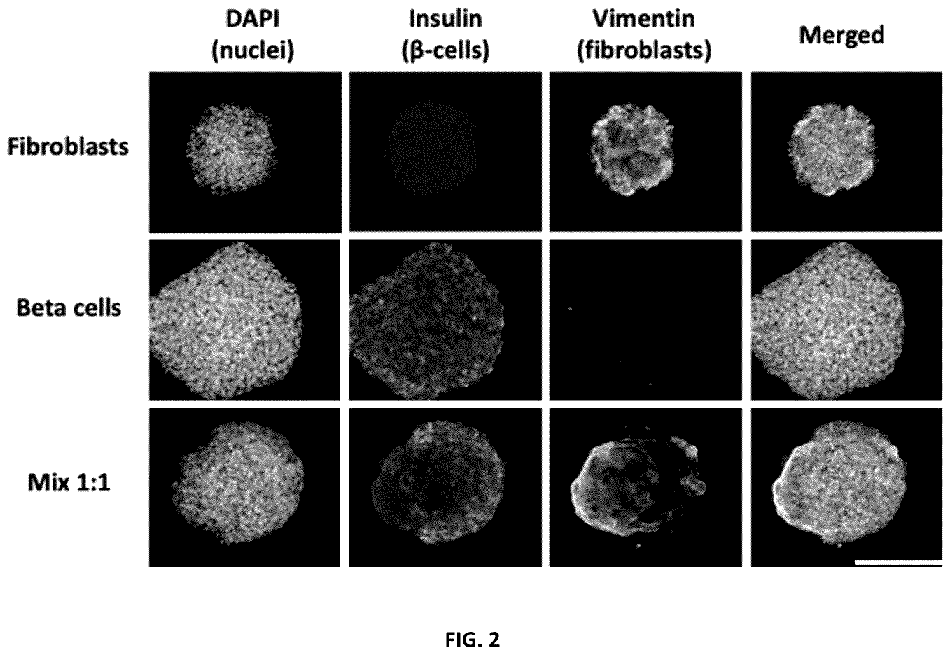

The results showed that mixed spheroids contained both cell types-fibroblasts and beta cells (FIG. 2). Beta cells showed production of insulin whereas fibroblasts did not (FIG. 2).

### Example 4—Insulin Production by Mixed Cell Spheroids

Spheroids comprising fibroblasts alone, beta cells alone, and a mixture of beta cells to fibroblasts at 1:4, 1:1, and 4:1 ratios were prepared as described in Example 1. Day 3 spheroids were used to study insulin secretion.

To study insulin production, spheroids were washed with phosphate buffered saline (PBS) and placed in starvation culture media overnight. On the next day, the spheroids were washed again with PBS and incubated in Krebs solution with 0.1% bovine serum albumin for 1 hour. After 1 hour the Krebs solution with albumin was replaced with fresh Krebs solution containing 20 mM glucose (stimulated condition) or Krebs solution without glucose (control condition). After 25 minutes, the supernatant was collected for quantification of insulin content using an ELISA assay (R&D Systems, Human/Porcine/Canine Insulin DuoSet).

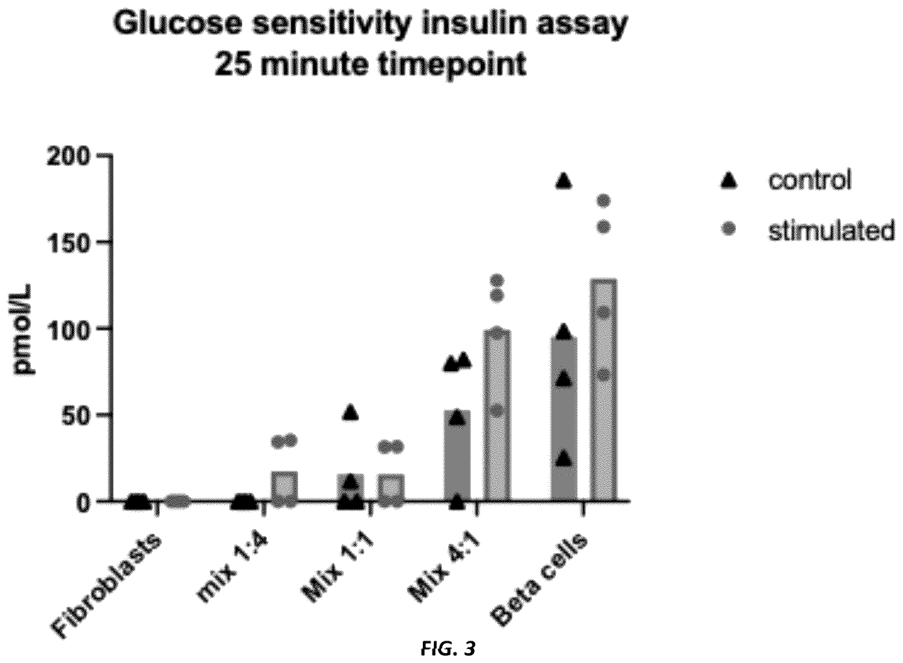

The results showed that mixed spheroids produce detectable levels of insulin compared to fibroblast only spheroids (FIG. 3). In addition, amount of insulin secretion by mixed spheroids was ratio dependent (FIG. 3).

### Example 5—Protection of Beta Cells by Fibroblasts

Spheroids comprising fibroblasts alone, beta cells alone, and a mixture of beta cells to fibroblasts at 1:4, 1:1, and 4:1 ratios were prepared as described in Example 1. Day 3 spheroids were used to study insulin secretion in the presence of innate immune cells.

To incubate spheroids in the presence of innate immune cells, a total of approximately 79 spheroids were collected from each condition (i.e., fibroblasts alone, beta cells alone, and a mixture of beta cells to fibroblasts at 1:4, 1:1, and 4:1 ratios) and placed into a separate tube per condition. On day 3, heparinized fresh whole blood from healthy rats was mixed with the spheroids from each condition and incubated overnight at 37° C. in a CO₂ incubator. After incubation, the tubes were spun in a centrifuge at 200×g, mixed with red blood cell lysis buffer, washed again, and then used to assess insulin production.

To assess insulin production by spheroids after exposure to innate immune cells, the spheroids were incubated in Krebs solution with 0.1% bovine serum albumin for 1 hour. After 1 hour the Krebs solution with serum was replaced with fresh Krebs solution containing 20 mM glucose. The supernatant from each condition was collected at various timepoints and used to assess insulin production over time using an ELISA assay (R&D Systems, Human/Porcine/Canine Insulin DuoSet).

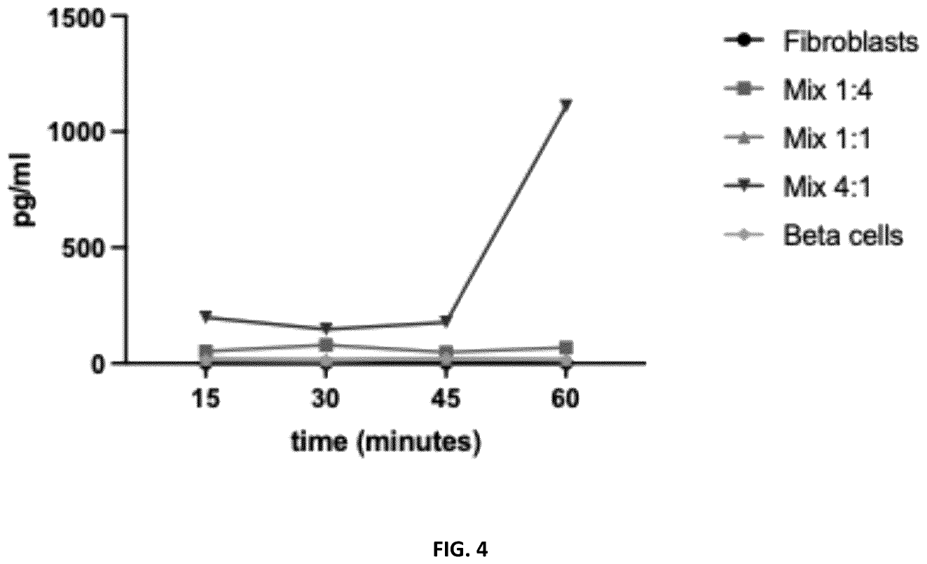

The results showed that only beta cells mixed with fibroblasts produced insulin (FIG. 4). These results suggested that encapsulation of pancreatic beta cells by fibroblasts con protect the beta cells from damage by immune cells.

All of the methods disclosed and claimed herein can be made and executed without undue experimentation in light of the present disclosure. While the compositions and methods of this invention have been described in terms of preferred aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The references cited herein, to the extent that they provide exemplary procedural or other details supplementary to those set forth herein, are specifically incorporated herein by reference.
1. atlas, I.d., IDF diabetes atlas, in IDF Diabetes Atlas, 10th edn. Brussels, Belgium: International Diabetes Federation. 2021.
2. Banday, M. Z., A. S. Sameer, and S. Nissar, Pathophysiology of diabetes: An overview. Avicenna J Med, 2020. 10 (4): p. 174-188.
3. Gregory, G. A., et al., Global incidence, prevalence, and mortality of type 1 diabetes in 2021 with projection to 2040: a modelling study. Lancet Diabetes Endocrinol, 2022. 10 (10): p. 741-760.
4. Global, regional, and national burden of diabetes from 1990 to 2021, with projections of prevalence to 2050: a systematic analysis for the Global Burden of Disease Study 2021. Lancet, 2023. 402 (10397): p. 203-234.
5. Buse, J. B., et al., 100 years on: the impact of the discovery of insulin on clinical outcomes. BMJ Open Diabetes Res Care, 2021. 9 (1).
6. Barrett, R. L. and E. Puré, Cancer-associated fibroblasts and their influence on tumor immunity and immunotherapy. Elife, 2020. 9.
7. Kennel, K. B., et al., Cancer-Associated Fibroblasts in Inflammation and Antitumor Immunity. Clin Cancer Res, 2023. 29 (6): p. 1009-1016.
8. Lefler, J. E., et al., STAT3 in tumor fibroblasts promotes an immunosuppressive microenvironment in pancreatic cancer. Life Sci Alliance, 2022. 5 (11).
9. Ping, Q., et al., Cancer-associated fibroblasts: overview, progress, challenges, and directions. Cancer Gene Ther, 2021. 28 (9): p. 984-999.
10. Sahai, E., et al., A framework for advancing our understanding of cancer-associated fibroblasts. Nat Rev Cancer, 2020. 20 (3): p. 174-186.
11. Wang, Z., et al., Cancer-Associated Fibroblasts Suppress Cancer Development: The Other Side of the Coin. Front Cell Dev Biol, 2021. 9: p. 613534.
12. Cords, L., et al., Cancer-associated fibroblast classification in single-cell and spatial proteomics data. Nat Commun, 2023. 14 (1): p. 4294.
13. Minini, M. and L. Fouassier, Cancer-Associated Fibroblasts and Extracellular Matrix: Therapeutical Strategies for Modulating the Cholangiocarcinoma Microenvironment. Curr Oncol, 2023. 30 (4): p. 4185-4196.
14. Ferrara, B., et al., The Extracellular Matrix in Pancreatic Cancer: Description of a Complex Network and Promising Therapeutic Options. Cancers (Basel), 2021. 13 (17).
15. Nissen, N. I., M. Karsdal, and N. Willumsen, Collagens and Cancer associated fibroblasts in the reactive stroma and its relation to Cancer biology. J Exp Clin Cancer Res, 2019. 38 (1): p. 115.
16. Xiao, W., et al., Matrix stiffness mediates pancreatic cancer chemoresistance through induction of exosome hypersecretion in a cancer associated fibroblasts-tumor organoid biomimetic model. Matrix Biol Plus, 2022. 14: p. 100111.
17. Linares, J., et al., Determinants and Functions of CAFs Secretome During Cancer Progression and Therapy. Front Cell Dev Biol, 2020. 8: p. 621070.
18. Cayabyab, F., L. R. Nih, and E. Yoshihara, Advances in Pancreatic Islet Transplantation Sites for the Treatment of Diabetes. Front Endocrinol (Lausanne), 2021. 12: p. 732431.

1. A composition comprising:
(a) one or more types of cells encapsulated by a plurality of cancer-associated fibroblasts (CAFs) and/or fibroblasts; and/or
(b) one or more types of cells encapsulated by extracellular matrix (ECM) secreted from CAFs and/or fibroblasts.

2. The composition of claim 1, wherein the encapsulated cells are pancreatic beta cells.

3. The composition of claim 1, wherein the encapsulated cells secrete a desired protein, enzyme, or metabolite, optionally a desired extracellular matrix protein secreted by a fibroblast or cancer associated fibroblast.

4. The composition of claim 1, further comprising one or more fibroblast-derived materials.

5. The composition of claim 4, wherein fibroblast-derived materials comprise exosomes, lysates, membranes, apoptotic bodies, or a mixture thereof.

6. The composition of claim 4, wherein the source of the fibroblast-derived materials is CAFs.

7. The composition of claim 4, wherein the source of the fibroblast-derived materials is fibroblasts that are other than CAFs.

8. (canceled)

9. (canceled)

10. The composition of claim 1, wherein the CAFs are engineered using gene editing to secrete ECM.

11. A method of generating the composition of claim 1, wherein at least some of the encapsulated cells are generated as one or more spheroids in a pipette tip, microwell, or microcavity.

12. The method of claim 11, wherein the CAFs, fibroblasts, and/or ECM is withdrawn into the pipette tip comprising the spheroid(s).

13. (canceled)

14. (canceled)

15. The method of claim 11, wherein prior to, during, and/or following generation of the composition, the CAFs and/or fibroblasts encapsulating the beta cells are treated ex-vivo to secrete ECM.

16. The method of claim 15, wherein the CAFs and/or fibroblasts are treated with one or more of hypoxic conditions, oxidative stress, and/or one or more growth factors produced by tumor cells.

17. The method of claim 16, wherein the growth factors comprise one or more of TGF-β, epidermal growth factor (EGF), fibroblast growth factor type 2 (FGF2), PDGF, Activin A, Nodal, or one or more BRAF inhibitors.

18. (canceled)

19. The method of claim 11, wherein prior to, during, and/or following generation of the composition, the CAFs and/or fibroblasts are activated with cytokines, chemokines, growth factors, transcription factors, and/or nucleic acids to secrete ECM.

20. The method of claim 11, wherein prior to, during, and/or following generation of the composition the CAFs and/or fibroblasts are activated with CAF-derived materials to secrete ECM.

21. The method of claim 20, wherein the CAF-derived materials comprise exosomes, lysates, extracellular vesicles, membranes or a combination thereof.

22. The method of claim 11, wherein prior to generating the CAFs are engineered to secrete ECM.

23. A method, comprising administering to an individual in need thereof the composition of claim 1 and/or CAF and/or fibroblasts exosomes, CAF and/or fibroblasts lysates, and/or CAF and/or fibroblasts extracellular vesicles.

24. (canceled)

25. The method of claim 23, wherein the encapsulated cells are pancreatic beta cells.

26. The method of claim 25, wherein the individual has diabetes.

27. The method of claim 23, wherein the individual has a metabolic disorder, phenylketonuria, tyrosinemia, homocystinuria, non-ketotic hyperglycinemia, maple syrup urine disease, amyloidogenic disorders, inherited cataracts, atherosclerosis, hemodialysis-related disorders, short-chain amyloidosis syndrome, achondroplasia, Morquio A syndrome, mucopolysaccharidosis I, CLN2 disease, maroteaux-Lamy syndrome, Alternating hemiplegia of childhood (AHC), Hydrops ectopic calcification-moth-eaten (HEM), Alzheimer's disease, Parkinson's disease, Huntington's disease, Creutzfeldt-Jakob disease, cystic fibrosis, Gaucher's disease, Tay-sachs, or Fanconi anemia.

28. A kit, housed in a suitable container, comprising the composition of claim 1 and/or CAF and/or fibroblast exosomes, CAF and/or fibroblast lysates, and/or CAF and/or fibroblast extracellular vesicles.

29. The kit of claim 28, further comprising one or more apparatuses to generate and/or administer the composition and/or CAF and/or fibroblast exosomes, CAF and/or fibroblast lysates, and/or CAF and/or fibroblast extracellular vesicles.

30. The kit of claim 29, wherein the apparatus comprises a pipettor, pipette tips, microwell plates, microcavity plates, and/or a syringe.
